(19) United States
(12) Patent Application Publication
(10) Pub. No.: US 2022/0071878 A1
(43) Pub. Date: Mar. 10, 2022

(54) ACIDIC STICK ANTIPERSPIRANTS AND DEODORANTS

(71) Applicant: Lume Deodorant, LLC, Chaska, MN (US)

(72) Inventors: Jeffrey Dean Lindsay, Appleton, WI (US); Shannon Klingman, Chaska, MN (US)

(21) Appl. No.: 17/486,813

(22) Filed: Sep. 27, 2021

Related U.S. Application Data
(63) Continuation of application No. 17/238,102, filed on Apr. 22, 2021.
(60) Provisional application No. 63/055,305, filed on Jul. 22, 2020.

(51) Int. Cl. A61K 8/44; A61K 8/67; A61K 8/92; A61K 8/36; A61K 8/34; A61K 8/891; A61K 8/73; A61K 8/24; A61K 8/41; A61K 8/46; A61Q 11/00; A61Q 15/00

(52) U.S. Cl. CPC A61K 8/447; A61K 2800/805; A61K 8/922; A61K 8/361; A61K 8/345; A61K 8/891; A61K 8/732; A61K 8/24; A61K 8/416; A61K 8/466; A61Q 11/00; A61Q 15/00; A61K 8/676; A61K 2800/874; A61K 2800/30; A61K 8/673

(57) ABSTRACT

Products and methods are disclosed relating to a deodorant stick comprising mandelic acid.

### BACKGROUND

### Field of the Invention

This invention pertains to products and methods for personal care, particularly personal care compositions and methods of use for reducing or preventing unwanted perspiration or odor associated with perspiration, particularly with an acidic stick.

### Description of Related Art

Many people seek to avoid the embarrassment or discomfort associated with perspiration or associated odors. Bacteria such as Corynebacteria feed off materials in the sweat, particularly the apocrine sweat glands found under the arms and elsewhere, and produce unpleasant odors. Many people purchase antiperspirants or deodorants for underarm use, for example, to mask odors or reduce perspiration. Rather than relying on fragrance to mask odor or on metal-containing compounds such as aluminum and zirconium salts to plug up sweat pores, novel approaches have been developed that can reduce odor by adjusting the skin microbiome, the array of microbes that live on the skin. Two patents of Shannon Klingman, U.S. Pat. No. 9,566,223, “Antiperspirants and Deodorants,” issued Feb. 14, 2017, and U.S. Pat. No. 8,992,898, “Antiperspirants and Deodorants,” issued Mar. 31, 2015, both of which are hereby incorporated by reference, describe the use of alpha hydroxy acids, particularly mandelic acid, in combination with caffeine for control of the skin microbiome as a natural deodorant that can dramatically reduce odor without requiring the use of aluminum or zirconium salts, and a third from the same inventor, U.S. Pat. No. 9,668,948, “Products and Methods for Reducing Malodor from the Pudendum,” issued Jun. 6, 2017, hereby incorporated by reference, describes compositions with mandelic acid for controlling odor from the pudendum. A related line of deodorant products marketed by Lume Deodorant, LLC of Chaska, Minneapolis (lumedeodorant.com) have captured a significant portion of the natural deodorant market with its oil/water emulsion products containing mandelic acid.

After development and commercialization of the Lume Deodorant cream with its combination of mandelic acid for adjusting the skin microbiome and caffeine for several other benefits including some degree of vasoconstriction for reduced perspiration, we recognized a need for a solid stick version of this product. However, professional formulators warned that its low pH would defeat the adaptation of typical approaches because the required emulsifiers or other components would not interact well with the low pH.

Unfortunately, conventional formulations suitable for natural deodorants in stick form faced significant challenges since the low pH range desired for the deodorant product and the amount of free mandelic acid required would either cause instability in the preparation due to incompatibility of many emulsifiers and other agents with low pH (e.g., from about 3 to 4.5 or from 3 to 4 or from 3.2 to 3.9, etc.), or result in skin irritation with encapsulated or isolated mandelic acid in a heterogeneous mixture since pockets of elevated acid content could cause small regions of irritated skin.

When a candidate stick was attempted with mandelic acid dispersed in water and combined with an oil phase, there appeared to be local pockets of high acid concentration in the solidified material that could irritate the skin. After further tests in which precipitated mandelic acid could be felt as a grit in the final solid, it seemed, without wishing to be bound by theory, that a key part of the problem may have been the precipitation of solid mandelic acid from a concentrated aqueous phase to form relatively large crystals that could either be felt as grit or could result in local irritation of the skin. These problems have contributed to the long-standing, unmet need in the market place for suitable high-performance acidic deodorant sticks. Such problems have now been overcome in inventive work described herein. Commercial methods for making antiperspirants are often anhydrous or employ high amounts of water as in gels or gel-like solids, such as having at least 20% water or at least 30% water. Such systems may employ silicones, water gels plus silicones, or lipids, but rarely combine silicones, lipids, and aqueous phases, where the formulating challenges can be severe. In a stick comprising a large portion of lipids and/or silicone compounds, it can be desirable to have a relatively small aqueous phase such as less than 30% by weight of the aqueous phase, or less than 25% water, more specifically less than 20%, 15%, 10%, 9%, 8%, 5%, Aol/o 7 and 3% water, such as from 1% to 20% water or 1% to 15% water or 1% to 12% water, 1% to 9% water, 1% to 5% water, or 0.5% to 3% water. This may be a desirable goal for reasons such as providing good tactile and aesthetic properties, as well as for product stability, reduced risk of moisture loss, more successful manufacturing due to a smaller water phase in making an emulsion, etc. However, with a small aqueous phase, the need to provide adequate levels of mandelic acid for effective odor control seems to require high levels of solids in a heated aqueous phase that could, upon cooling, result in precipitation of the solids and the formation of grit and/or pockets of concentrated mandelic acid that might be irritating and/or have undesirable tactile properties. Caffeine or other solids may also precipitate and contribute to tactile issues. Thus, both expert external opinion and our own experimental work suggested that a solid stick with relatively low water content and high mandelic acid content was not possible by simply adapting standard approaches known in the art and may not even be possible at all if it were to be mass produced at an affordable price while still providing good performance, good tactile properties, and low risk of skin irritation.

Surprisingly, we have discovered that successful, high-performance acidic deodorant sticks can be made at low pH and with good tactile properties using innovative approaches described herein.

Commercial methods for making antiperspirants are often anhydrous or employ high amounts of water as in gels or gel-like solids, such as having at least 20% water or at least 30% water. Such systems may employ silicones, water gels plus silicones, or lipids, but rarely combine silicones, lipids, and aqueous phases, where the formulating challenges can be severe. However, we have discovered that successful, high-performance acidic antiperspirant sticks can be made as inventive variations of certain principles from Applicant's inventive acidic deodorant sticks, wherein antiperspirant sticks at low pH and with good tactile properties can be provided using, for example, relatively low-moisture formulations with silicones and lipids or substantially anhydrous formulations involving lipids and/or silicones. Further, we have discovered that such sticks can be made acidic not only with alpha-carboxylic acids such as mandelic acid or lactic acid, but can also have desirable effects on the skin and skin microbiome by including another acidic material, N-acetyl cysteine, which may be useful in hindering the growth of biofilms and of undesirable bacteria while also having a variety of potential positive effects on skin.

However, the problems that were overcome creating acidic deodorant sticks are even more challenging when attempting to create acidic antiperspirant sticks such as those comprising aluminum and zirconium compounds approved for antiperspirant use. Inventive methods for creating an acidic stick, as described herein, face added challenges when it was desired to add significant antiperspirant materials, for when a relatively small amount of water is used to dissolve mandelic acid or other acids, the water may interact adversely with some of the water-soluble antiperspirant material. For example, since the water may not be abundant enough to dissolve all of the antiperspirant particles, often in powder form, and since any dissolved antiperspirant may compete with other dissolved solids and contribute to precipitation problems, there may be complex interactions and poor stability or poor texture or poor performance. Fortunately, these problems were also overcome with further inventive work, resulting in both substantially anhydrous and hydrous formulations to achieve low pH, suitable physical properties, and useful levels of antiperspirant materials in the stick.

### SUMMARY

Applicant has found that successful acidic antiperspirant sticks can be made in compositions comprising lipids or lipids and silicone compounds by combining an oil, silicone, or oil-silicone phase with a relatively viscous acidic phase comprising one or more acidic components such as mandelic acid or other alpha-hydroxy acids and N-acetyl cysteine, wherein a the viscous acidic phase has a viscosity substantially greater than that of water, such as at least 5 times higher (e.g., at least 5 centiStokes or cst). The acidic thickener such as an aqueous starch mixture, a polyol such as propane diol, gums or water-swellable polymers or minerals dispersed in water, etc., is present in the aqueous phase, making it substantially more viscous than water. Such a thickened acidic aqueous phase can be combined with a heated oil phase or oil-silicone phase in the presence of suitable emulsifiers or gelling agents, after which optional agents such as dry powders (e.g., starch, silica materials, silicone powders such as polymethylsilsequioxane, etc.) can be combined with the emulsion or blend of an aqueous phase and an oil or oil-silicone phase, along with other finishing agents such as preservatives, fragrances, other silicone liquids, volatile materials such as volatile silicones, etc.

In some embodiments, the resulting acidic stick has from 10% to 50% lipids, from 10% to 40% silicones, from 5% to 25% starches or starch derivatives, from 0.5% to 10% acidic agents such as mandelic acid and N-acetyl cysteine (NAC), and may have less than 20%, 15%, or 10% water. The stick may be formed by providing the acidic components in a thickened aqueous phase having a viscosity of at least 5 cps at 25° C. which is heated and blended into an oil phase or oil-silicone phase or silicone phase, followed by blending in of additional components, including solid powders or finishing agents.

Thus, in one embodiment, a novel acidic stick deodorant is provided comprising a personal care composition comprising a solid or semi-solid waxy phase comprising one or more waxes and/or other lipids, a starch or starch derivative, a thickener, and at least 1′)/0 of an alpha-hydroxy acid associated with the thickener, wherein the alpha-hydroxy acid is substantially uniformly dispersed in the stick. The acidic stick may, in one embodiment, have from 0.2% to 20% water (e.g., from 3% to 15%), 1′)/0 to 9% alpha hydroxy acid, 5% to 35% silicone compounds, 10% to 40% lipids, 0.5% to 10% emulsifiers or gelling agents, and 2% to 20% starch or starch derivatives. It may also comprise from 0.5% to 10% of a polyol.

We have therefore found that successful acidic deodorant sticks can be made in relatively low-moisture formulations with silicones and lipids or substantially anhydrous formulations, achieving high levels of acidic ingredients such as mandelic acid and/or N-acetyl cysteine, without creating compositions that can are readily perceived as gritty. In particular, we have found that certain techniques such as significantly elevating the viscosity of the aqueous phase, in combination with carefully selected ingredients and other innovative formulation methods, can overcome multiple problems that hindered the development of a successful deodorant stick with high mandelic acid content. Through the approaches and formulations described herein, we have found that high levels of mandelic acid and other solids can be present in a minor aqueous/polar phase that, when combined with an oil/non-polar phase (including silicone or oil-silicone phases), can subsequently be cooled to room temperature without leading to the formation of perceptible grit and without evidence of skin irritation from nonuniformity in the distribution of the acidic components. In some embodiments, there may be no perceptible indication of alpha hydroxy acid precipitation at all, and if particles do precipitate, they may be so fine as to provide virtually no tactile clue of their existence. The resulting solid can have a luscious, smooth feel that can be applied comfortably and can be used with successful odor control without undue risk of skin irritation, functioning much like the thoroughly proven and successful Lume® Deodorant cream.

Surprisingly the approaches described herein seem to be able to solve multiple problems at once, including one or more of: a) difficulties in forming a stable dispersion involving oil and water phases at low pH, b) the problem of skin irritation from large pockets of alpha hydroxy acid in the cooled stick, c) the problem of poor texture due to a gritty feed from precipitated solids that were once in or largely in the aqueous phase, and d) the difficulty of having the acidic components sufficiently accessible to the skin to be able to modify the skin microbiome and/or effectively reduce malodor from certain bacteria. Note, however, that the ability of some embodiments to overcome more than one obstacle or to provide more than one benefit should not be taken as a requirement that all embodiments within the scope of the invention as claimed must necessarily solve the same plurality of problems of provide the same plurality of benefits.

Applicant's novel method and formulation approach has overcome previous barriers, allowing creation a novel low-pH stick with high odor control performance and excellent tactile properties in a smooth, easy-to-use, substantially uniform deodorant stick.

Further, we have discovered that acidic sticks can be made acidic not only with alpha-carboxylic acids such as mandelic acid or lactic acid, but are believed to potentially have additional positive effects on the skin and skin microbiome by including another acidic material, N-acetyl cysteine, which may be useful in hindering the growth of biofilms and of undesirable bacteria while also having other skin health benefits.

Surprisingly, we have found that by significantly elevating the viscosity of the aqueous phase, in combination with carefully selected ingredients and innovative formulation methods, we can overcome multiple problems that hindered the development of a successful deodorant stick with high mandelic acid content. Surprisingly, through the approaches and formulations described herein, we have found that high levels of mandelic acid and other solids can be present in a minor aqueous/polar phase that, when combined with an oil/non-polar phase (including silicone or oil-silicone phases), can subsequently cool without leading to the formation of perceptible grit and without evidence of skin irritation from nonuniformity in the distribution of the acidic components. The resulting solid can have a luscious, smooth feel that can be applied comfortably and can be used with successful odor control without undue risk of skin irritation.

We have therefore found that personal care deodorant and antiperspirant compositions comprising effective levels of alpha hydroxy acids such as mandelic acid and/or other acidic materials can be formulated in a solid stick for convenient application to the underarms or other regions of the body. Such sticks can also comprise caffeine and may be substantially aluminum free and zirconium free or may comprise significant amount of aluminum and/or zirconium compounds, such as at least 3% by weight, at least 5%, 8%, 10%, 12%, or 13%, such as from 3% to 30%, 3% to 25%, 3% to 20%, 5% to 24%, etc. In some embodiments, the composition is prepared by combining one or more acidic materials such as mandelic acid in a solvent to create an “acid paste” having a viscosity substantially greater than water such as at least 5, 50, 100, or 200 times greater than water. The acid paste may comprise water, water and one or more polar solvents, or a polar organic solvent other than water, with an thickener such as a starch, a gum, minerals such as laponite, polymers such as a polyacrylate or carbomer, high-viscosity polar solvents, or other agents that swell in water or other solvents, or combinations thereof. The resulting viscous acidic mixture is, as defined herein, an “acid paste” that can then be combined with a non-aqueous phase (an oil phase and/or a silicone phase) to form an emulsion or other mixture that can be cooled to form a solid stick, optionally after adding further ingredients such as fragrances, powders (e.g., starch, silica, silicone materials, and other solids), liquids (e.g., esters, alcohols, or silicone liquids such as an alkyl silicone liquid), or other agents. The acid paste when added to the other ingredients, prior to any evaporation of water during mixing, may comprise from 1% to 35% of the mixture, such as from 3% to 25%, 3% to 20%, 3% to 15%, and 5% to 17%.

For example, we have found that an aqueous solution of mandelic acid or other soluble acidic materials can be used, such as a solution comprising from 2% to 40% total of mandelic acid and/or N-acetyl cysteine in water at a suitable temperature, or water combined with other polar solvents such as 1-2 propanediol, 13 propanediol, glycerin, ethanol, propylene carbonate, or other alcohols, glycols, or esters, or in some embodiments, in a solution of such polar organic solvents that may be substantially free of water. In some cases, the solution may be supersaturated or substantially saturated.

The viscosity of the acid solution can be elevated to be substantially greater than that of water using a thickener such as a gum, a starch (corn starch, tapioca starch, potato starch, cassava, arrowroot starch, chemically modified starches such as modified food starch, cold-water soluble starches, and the like), a polymer such as a polyacrylate or copolymers thereof or known superabsorbent polymers, hydroxymethyl cellulose or other water soluble cellulosic derivatives, water-swellable polyurethanes such as those described in WO2004029125A1, etc. Thickener levels relative to the solvent mass may range from, for example, 0.1 to 15 weight percent, such as at least 0.3, 0.5, 1, 2, 3, 4, 5, or 6 weight percent, up to one of any suitable integer from 2 to 15 weight percent, from 2 to 10 wt %, from 2 to 6 wt %, etc. When starch is used, the starch and solvent, which may comprise water or a polar organic solvent, is then heated until the starch grains swell (gelatinize) and cause the slurry to become a thickened paste. In water-starch slurries, this may occur between about 50° C. and 80° C., for example, with many native starches tending to gel around 60° C. to 71° C. Rather than gelling with the addition of heat, a soluble starch may be used that is soluble in cold water. The resulting acidic starch paste has elevated viscosity and reduced opacity relative to the initial slurry. An appropriate amount of this slurry, which may be heated to temperature from 40° C. or 50° C. to 70° C. or 80° C., for example, and can then be combined with a molten waxy phase or oil-silicone phase to create a dispersion or emulsion that does not readily separate. Emulsifying waxes, other emulsifiers, or gel-producing agents such as those comprising hectorite particles or other minerals may be present but need not be used in some embodiments. Gel-making agents with hectorite may be particularly useful when combining silicone oils with lipids and the acid paste.

The dispersion may be continually stirred or otherwise blended using rotary mixers, whisks, homogenizers, static mixers, etc., and if desired may be kept at an elevated temperature for a suitable time to promote evaporation of some of the solvent or especially a portion of the water if desired. The dispersion may then be blended with additional agents such as powdered starch or other powders including laponite, talc, hydroxyapatite and derivatives thereof, magnesium hydroxide, magnesium stearate, zinc stearate, zinc oxide, other zinc compounds, antiperspirant salts such as aluminum and zirconium salts, silica, sillylated silica or other solids, microspheres, and the like. In some embodiments, however, the deodorant stick is substantially free of aluminum salts and/or substantially free of zirconium salts.

In another embodiment, a water/oil emulsion or dispersion comprising mandelic acid associated with an aqueous solvent in an aqueous phase is heated to drive off a portion of the water such as at least 20%, at least 40%, or at least 60% of the water, with exemplary ranges of 20% to 90% or 30% to 80%, resulting in a highly uniform distribution of mandelic acid throughout a waxy phase. Continued stirring may be applied during heating as water is driven off. The peak temperature of the mixture in this process may be at least 75° C., 80° C., 90° C., 95° C., 99° C., 100° C., 110° C., 115°, or in general any whole number between 75° C. and 150° C., such as from 85° C. to 140° C. or 85° C. to 130° C. or from 90° C. to 125° C. The composition may remain above 75° C., 80° C., 85° C., 90° C., 95° C., 99° C., 100° C. or 110° C. for at least 1 minute or any whole number of minutes from 1 to 90, such as from 1 minute to 30 minutes or for at least 2 minutes.

For example, mandelic acid may be dissolved in a mixture of water and an alcohol such as ethanol that may also comprise other soluble material or other solvents such as glycerin or propanediol, and this ethanol-water-acid mixture is combined with a mix of waxes, fatty acids, esters, butter, oils, and related lipids, optionally including at least one emulsifying wax or gel-producing agent. If no thickening agent is included, emulsifying waxes can be particularly helpful in promoting good mixing of the aqueous phase with the oil phase. As the combination is heated and stirred, the solids melt and begin to form a dispersion or emulsion with the ethanol-water-acid mix dispersed in the oil phase. As heating continues, water and ethanol may be progressively driven off, resulting in a waxy material with mandelic acid finely dispersed throughout. Prior to cooling, the melt may be combined with a starch such as arrowroot or tapioca starch to provide additional body and tactile properties, and may then be poured into a suitable container such as an oval-shaped deodorant container or mold.

Thus, in one embodiment, we have developed a personal care composition in the form of a deodorant stick for reducing at least one of perspiration and body odor, the stick comprising from 0.2% to 12% by weight of mandelic acid or other acidic materials particularly solids (when dry) distributed substantially uniformly throughout a solid or semi-solid waxy phase, such that the composition at room temperature is free of tangible or visible acid grains or crystals in a suitable carrier for application to the skin. In some embodiments, the acid such as mandelic acid is associated with starch granules dispersed throughout the stick that may be swollen, never swollen, or swollen and at least partially dried after contact with the mandelic acid. In some embodiments, the stick comprises two forms of starch, one that was previously gelatinized (swollen) in the presence of mandelic acid, and one that was added as unswollen particles to a molten waxy mix comprising previously the swollen starch particles associated with mandelic acid. Both the swollen and unswollen starch particles may independently comprise one or more starches such as corn starch, tapioca starch, pea starch, potato starch, cassava starch, and arrowroot starch.

In some embodiments, silicone materials such as cyclopentasiloxane, dimethicone, silica silylate, silica dimethyl silylate, trimethylsiloxysilicate and trifluoropropyldimethyl/Trimethylsiloxysilicate and other siloxanes can be present and may be combined with waxes and oils to impart slip or other tactile or rheological properties or to improve delivery when rubbed against the skin. The silicone content may be from 1 to 50%, such as from 1% to 40%, 5% to 40%, 3% to 36%, 1% to 25%, 8% to 39%, or from 11% to 40%. Silicone-treated silica, silicone-coated particles, silicone microspheres, and other particles comprising silicone may be considered as well.

In some embodiments, the composition may be substantially free of volatile silicone compounds such as volatile cyclic silicone oils, in particular cyclotrisiloxane, cyclotetrasiloxane, cyclopentasiloxane, and cyclohexasiloxane. Other volatile silicone oils that may be substantially excluded include linear or branched silicone oils including 2 to 10 siloxane units, such as hexamethyldisiloxane, octamethyltrisiloxane, and decamethyltetrasiloxane. In some embodiments, the composition may be substantially free of volatile silicone oils or any silicone oils having a viscosity at 25° C. of 5 cst or less, particularly silicone oils that do not comprise alkyl chains.

The composition may also be substantially free of volatile nonsilicone oils, such as any one or more of isodecane, isoundecane, isododecane, isotridecane, isotetradecane, isopentadecane, and isohexadecane, or may be substantially free of C8-C20 isoparaffins, C8-C18 isoparaffins, or C8-C16 isoparaffins.

Lipids such as waxes and oils used herein may have a smoking point of at least 160° C., 170° C., 180° C., 190° C., and 200° C., and alternatively may be within a range between any of the aforementioned smoking point temperatures and an upper limit of any integral value between 250° C. and 320° C. such as 280° C. or 300° C.

Caffeine or other xanthines may be present and may be applied by fully or partially dissolving caffeine in an oil phase or silicone-rich phase prior to combination with mandelic acid materials, or may be fully or partially dissolved in the mandelic acid solution or other aqueous phase to be combined with the oil phase. One approach that has yielded excellent results has been to combine the caffeine with the aqueous phase comprising the mandelic acid. Without wishing to be bound by theory, it is believed that the presence of the mandelic acid enhances the solubility of caffeine, which can be useful when relatively high levels of caffeine or relatively low levels of moisture are desired in the final product. Heating the aqueous phase with caffeine may be useful in enhancing solubility of caffeine or other materials, and thus the aqueous phase may be heated to, for example, from 40° C. to 110° C., from 50° C. to 100° C., from 50° C. to 90° C., or from 50° C. to 80° C. prior to combining with the oil phase and/or a silicone phase. The addition of other solvents such as propane diol, propylene glycol, glycerin, and other agents may also assist caffeine solubility.

We have also obtained good results in some cases by adding caffeine powder to the oil phase where a portion appears to dissolve in at least some cases, and then combining an acid mixture in an aqueous phase such as mandelic acid in a starch paste, a gum-based paste or gel, etc., wherein the caffeine ultimately appears to be dissolved or at least finely dispersed in the heated mixture. Without wishing to be bound by theory, the caffeine is believed to provide beneficial functions by serving as a vasoconstrictor that can help close pores to reduce sweating, but also by a protective or anti-irritant function that reduces the risk of skin irritation from the presence of mandelic acid or other acids on the skin. Caffeine may be present relative to the oil phase at a level of 0.1° A to 7%, from 0.3% to 5%, from 0.5% to 3%, and from 0.3% to 2.5%. Relative to the final composition, the caffeine concentration may be from 0.05% to 5%, from 0.1% to 4%, and from 0.3% to 3%. In some embodiments, however, less than 1% caffeine is present, less than 0.5% is present, or the composition may be substantially caffeine free.

Other xanthines are also believed or known to have vasoconstrictive effects relative to the skin or other potentially useful effects relative to antiperspirant and deodorant products, including pharmacological effects related to those of caffeine, and thus xanthines such as methylxanthines and derivatives thereof are considered within the scope of certain embodiments of the present invention. Methylxanthines include theophylline (1,3-dimethyl-7H-purine-2,6-dione, also known as dimethylxanthine), caffeine (1,3,7-trimethyl-1H-purine-2,6(3H,7H)-dione, also known as 1,3,7-trimethylxanthine or methyltheobromine), and theobromine (3,7-dimethyl-2,3,6,7-tetrahydro-1H-purine-2,6-dione, also known as 3,7-dimethylxanthine or xantheose). Derivatives of xanthine compounds including salts thereof may be used, including caffeine citrate and other salts with carboxylic acids.

Xanthine-containing herbs include Camellia sinensis (Tea), Coffea arabica (Coffee beans), Cola nitida (Kola), Cola acuminata, Theobroma cacao (Cacao), Theobroma bicolor, Theobroma angustifolium, Ilex paraguariensis (Mate), Pauffinia cupana (Guarana), Banisteriopsis inebrians, Davilla rugosa, Euonymus europaeus, Erodium spp., Genipa spp., Lippia multiflora, Maytenus spp., Sterculia spp., Tylophora moffissima, the Yaupon Holly (Ilex vomitoria) and Villarsia spp. The xanthine compound may be provided via a plant extract or mixture of plant extracts. In one embodiment, the composition comprises an herbal extract which has been substantially enriched in xanthine content beyond that available by forming an extract with heated water or ethanol alone. In one embodiment, for example, substantially pure caffeine and a xanthine-containing plant extract are combined to form a personal care composition with deodorant and/or antiperspirant properties.

The mass ratio of the carboxylic acid to total xanthine compounds may be any practical finite number such as from 0.1 to 5.0, from 0.5 to 30, from 1 to 10, or from 1 to 5, or substantially greater than 1 such as about 1.5 or greater.

The mandelic acid in the final composition may have a concentration of about 0.2% to 10%, from 0.5% to 8%, from 0.5% to 5%, and from 1% to 8%.

When a portion of the finished composition such as a pellet of mass 0.2 g is combined with twice the mass of liquid water and mixed or rubbed together at room temperature and placed in contact with a pH paper to estimate the pH, the apparent pH may be from 2 to 6, such as from 2.8 to 5.7, from 3 to 5.5, from 3.2 to 5.5, and from 3.5 to 5.4. A suitable pH paper is the Hydrion® pH 3.0-5.5 paper, as well as Hydrion® pH 0.0 to 6.0 paper.

Without wishing to be bound by theory, the role of mandelic acid or other suitable carboxylic acids is believed to be that of an acidifying composition that reduces the pH of the skin to a level that limits the growth of the bacteria that produce undesirable odors. Such bacteria can include Corynebacteria and Propionobacteria that dwell on the skin. In addition or alternatively, the reduced pH creates an environment that protects or maintains healthy microbial flora on the skin, thereby controlling the levels of less desirable bacteria that may produce unwa

### Definitions

As used herein, “deodorant” refers to compositions that are commonly used to reduce unwanted body odors associated with perspiration and/or bacteria on the surface of the skin. “Deodorants” may reduce odor through a variety of means, and such means in the various embodiments of the present invention may include suppression of bacterial activity, antimicrobial mechanisms, chemical interference with odor generation mechanisms, removal or modification of feedstuff for odor-producing bacteria, masking of odors, absorption of odorous materials, and the like.

As used herein, “antiperspirant” refers to materials that help reduce perspiration on the skin, and are often also relied on to reduce odor that may be generated by bacteria acting upon sweat. Many common antiperspirant agents act by forming plugs that block the pores associated with sweat glands or otherwise reducing the flow of sweat. A deodorant may function as an antiperspirant but need not do so to be a deodorant.

Antiperspirants recognized for use in the United States include aluminum chlorohydrate, aluminum chloride, aluminum zirconium trichlorohydrate, aluminum zirconium trichlorohydrex gly (a mixture of monomeric and polymeric Zr4+ and A13+ complexes with hydroxide, chloride and glycine), aluminum zirconium tetrachlorohydrate, aluminum zirconium tetrachlorohydrex gly, aluminum chlorohydrex polyethylene glycol, aluminum chlorohydrex propylene glycol, aluminum dichlorohydrate, aluminum dichlorohydrex polyethylene glycol, aluminum sesquichlorohydrate, aluminum sesquichlorohydrex polyethylene glycol aluminum sesquichlorohydrex propylene glycol aluminum zirconium octachlorohydrate, aluminum zirconium octachlorohydrex gly, aluminum zirconium pentachlorohydrate, aluminum zirconium pentachlorohydrex gly, aluminum zirconium tetrachlorohydrate, and aluminum zirconium tetrachlorohydrex gly, compounds which typically are allowed at levels up to 20 or 25 weight percent.

The compositions of various embodiments of the present invention may generally be described as deodorants and in many cases may generally be described as antiperspirants, though a composition that has limited antiperspirant efficacy is not necessarily outside the scope of the claimed invention, which is defined by the claims appended hereafter.

As used herein, “effective pH” refers to a measure of the pH of a solid or semi-solid deodorant material or related material when it is combined with distilled water. About 0.100 g (e.g., from 0.085 to 0.13 g) of the material is placed in a weighing dish and combined with a mass of distilled water equal to twice the mass of the matter being tested. The weighing dish should be the 7-ml volume plastic intermediate dish provided with the Smart Weight Gem50 jewelers balance (0-50 g, milligram precision digital scale). The material being measured is combined with water and then smeared in the dish by hand to contact the water thoroughly with the material, blending for about 10 seconds. After 15 more seconds, a pH paper strip is contact with the water phase to read the pH. The pH paper may be the Hydrion® 3.0 to 5.5 strip, the Hydrion® 0 to 6.0 strip, or the Lab Essentials™ Universal 1-14 pH Paper, relying on the paper with the smallest range that encompasses the pH of the material being measured. Note that with pH paper, as water wicks up the paper, its pH may change as acidic components are absorbed by or reacted with components in the paper, so the leading front of water wicking into the paper may display a more neutral pH than what is indicate in the main body of the wetted paper, so the pH near the leading edge of the wicking front should be disregarded. In some embodiments, the deodorant stick materials disclosed herein may have effective pH values less than 7, such as from 2 to 6.5 or any other pH range discussed herein or with upper or lower limits selected from any pH value discussed herein or in other references incorporated by reference.

As used herein, a “solvent” for dissolving mandelic acid, caffeine, or other solids includes water, aqueous solutions, or a variety of other suitable compounds alone or in combination with water or other solvents described herein, such as propylene carbonate, ethanol, propanol, butanol, 1-3 propanediol, 1-2 propanediol (also known as propylene glycol), 3-phenyl-1-propanol, (2,2-Dimethyl-1,3-dioxolan-4-yl)methanol (also known as Solketal, isopropylidene glycerol, or Augeo Multi Clean), glycerin, pentylene glycol, 2-methoxy-2-phenylethanol, and 2-phenylethanol. Such polar organic solvents may also be used without added water to create a low-water or substantially water-free acid paste. In some cases, a solvent can also be suitable as a thickener or part of a thickener composition.

As used herein, “acid paste” refers to an aqueous or polar/hydrophilic phase comprising mandelic acid that is combined with a non-aqueous/non-hydrophilic phase (e.g., an oil phase or silicone phase) in the methods for preparing an acidic deodorant stick disclosed herein. The “acid paste” is a relatively viscous aqueous solution or gel of an acid such as a carboxylic acid, particularly mandelic acid, and generally comprises water, at least one carboxylic acid such as mandelic acid, one or more thickeners and/or relatively viscous polar organic solvents, and optionally a base or buffering agents (e.g., salts such as sodium hydroxide or potassium hydroxide, bicarbonates, carbonates such as sodium carbonate, and the like) to bring the effective pH of the final product to a desired level. The acid paste may also comprise caffeine or other agents that are more readily dissolved or dispersed in an aqueous or polar/hydrophilic phase than in an oil or oil-silicone phase.

While a dictionary definition of “paste” can be “a thick, wet substance used for sticking things together, or any soft, wet mixture of powder and liquid” (//dictionary.cambridge.org/us/dictionary/english/paste), the term “paste” as used herein was initially selected based on the successful use of pastes made with starch powder and water combined with mandelic acid and cooked to gelatinize or at least partially gelatinize the starch to form a viscous paste. Gelatinized starch in water is typically described as a paste and is a suspension of swollen starch vesicles suspended in water, not actually a solution nor a colloid, though often mislabeled as such. However, the term “acid paste” as defined herein can more generally to other thickened aqueous or hydrophilic preparations that are substantially more viscous than water, including aqueous preparations made with starches, flours, gums, superabsorbent or swellable polymers such as polyacrylates or acrylic cross polymers or cellulose derivatives, swellable minerals such as laponite, viscous fluids such as glycerin or propylene glycol, and the like.

Acid pastes are generally aqueous but need not have substantial water content. In some embodiments, other hydrophilic solvents can be used. For example, we have demonstrated that 1-3 propane diol, glycerin, and propylene glycol can all be used to dissolve both mandelic acid and caffeine at useful concentrations for preparing a deodorant stick with useful levels of mandelic acid and optionally caffeine. For example, we have demonstrated that 5 ml of these compounds can successfully dissolve 0.5 g of mandelic acid and 0.2 g of caffeine, giving a simple “acid paste” with nearly 10% mandelic acid and 4% caffeine. Elevated temperature (generally above 60° C.) was required to dissolve all of the solids. If used as roughly ⅓ the mass of a deodorant stick with additional lipids, silicones, and other ingredients, the final product, would have over 3% mandelic acid and 1% caffeine and be essentially water free. Thus, the acid paste may comprise a polar organic solvent such as propane diol or propylene carbonate and optional water comprising mandelic acid, optionally additional thickening agents, optionally caffeine, optionally basic salts to adjust pH, and other agents as needed.

“Relatively more viscous than water” as used herein to describe the acid starch indicates that it is at least 5 times, 10 times, 50 times, 100 times, or 500 times more viscous than water. Alternatively, the measured viscosity may be at least 5, 10, 50, 100, or 500 centipoise (cps), such as from 5 to 100,000 cps, from 10 to 50,000 cps, from 100 to 25,000 cps, from 100 to 5,000 cps, from 50 to 5000 cps, etc. The viscosity can be measured at 25° C., or, alternatively, at 60° C. or 70° C., with a Brookfield viscometer operating at 20 RPM. For an acid paste comprising starch or other solids not yet dissolved, viscosity should be measured only after the acid paste has first been heated sufficiently (e.g., to at least 70° C. or 75° C.) to gelatinize the starch or to promote more complete dissolving of solids before bringing the acid paste to the chosen temperature for viscosity measurement. Unless otherwise specified, the temperature for viscosity measurement of the paste can be taken as 70° C., which can in many embodiments also be a useful temperature for blending the aqueous phase with the oil phase and/or silicone phase, if present. Without wishing to be bound by theory, it is believed that the elevated viscosity of the aqueous phase relative to water assists in the blending of the aqueous phase with the relatively more viscous oil and/or silicone phases (including an oil phase already comprising silicone compounds), and/or promotes stability of the resulting emulsion or mixture. The viscosity of the acid paste (aqueous phase) may be measured using a Brookfield viscometer, model RVF, at 20 rpm, following directions at brookfieldengineering.com/-/media/ametekbrookfield/manuals/obsolete%20manuals/dial%20m85-150-p700.pdf?la=en.

As used herein, a “thickener” is an agent that can substantially increase the viscosity of a solution of mandelic acid in a solvent. Thickeners may include starches such as native starches, modified starches, cold-water soluble starches, and the like, including but not limited to corn starch, tapioca starch, potato starch, cassava starch, arrowroot starch, wheat flour, sago, cationic starches such as cationic corn starch, etc. Thickeners may also include gums such as xanthan gum, guar gum, Sclerotium gum, locust bean gum, acacia gum, konjac gels, alginin and its derivatives, namely, alginic acid, sodium alginate, potassium alginate, ammonium alginate, and calcium alginate, and the like. Polysaccharide gums and other polysaccharide thickeners may be used such as pullulan, pectin, agar, gelatin, and carrageenan (both kappa and iota forms) can be considered. Cellulose derivatives may also be used such as cellulose ether derivatives such as carboxymethyl cellulose, methyl cellulose, ethyl cellulose, ethylmethyl cellulose, hydroxyethyl cellulose, hydroxymethyl cellulose, hydroxyethylmethyl cellulose, hydroxypropyl cellulose, hydroxypropyl methyl cellulose, hydroxypropyl ethyl cellulose, hydroxyalkylcellulose polymers, and the like. Mineral agents may be used such as slurries of clay materials such as kaolin, hectorite, thickening waxes sold for cosmetic purposes, betonite, laponite, silica, alumina, attapulgite, montmorillonite, hydroxyapatite, talc, etc., and mixtures or derivatives thereof. Various polymers may also be used such as polyvinyl alcohol, polyacrylic compounds such as carbomer, polylactic acid, carboxomer polymers, various superabsorbent polymers, and the like. Viscous polyols such as those having a viscosity at least 5 times or at least 50 times that of water may be considered. In some embodiments, however, the thickener itself may be substantially free of polyols, though polyols may be present as a solvent.

As used herein, polyols, also known as polyhydric alcohols, are defined as organic compounds having at least two hydroxyl groups per molecule. The general formula of the suitable polyols are: R(OH), where n is equal to or greater than 2 and R is generally C2-C10 alkyl or substituted alkyl group. Suitable polyols may include glycerin (also known as glycerol), propylene glycol (also known as 1,2-propanediol), 1,3-propanediol, 1,2-butanediol, 1,3-butanediol, 1,2-pentanediol, 1,5-pentanediol, isopentyldiol, 1,2-hexanediol, 1,6-hexanediol, diethylene glycol, diglycerin, dipropylene glycol, triethylene glycol, 1,2,3-hexanetriol, 1,2,6-hexanetriol, or combinations of the suitable polyols in any given ratio. 1,6-Hexanediol, also known as hexamethylene glycol, is a solid at room temperature (melting point: 42.8° C.) and may also be considered. In general, any alkyl diol having from 3 to 9 carbons and a viscosity at 20° C. of at least 20 mPa-s and more specifically any 1,n-alkanediols for n less than 9 may be considered. Liquid alkyl triols may be considered such as butanetriol. Esters of alkyl glycols (mono- and diglicerides, for example) having up to 7 carbons esterified with carboxylic acids or fatty acids, the acid having up to 8 carbons, may be considered, including, for example, neopentyl glycol diheptanoate, provided the melting point of the ester is no higher than 80° C., 70° C., 60° C., 50° C., 40° C., 30° C., or 20° C. In some embodiments, the thickener can act as a solvent. In some such embodiments, the thickener, when heated to 70° C. in substantially pure form, can dissolve mandelic acid powder at a level of at least 20 g, 15 g, 10 g or 5 g per 100 g of thickener. Polyols, whether used as a thickener or a solvent or both, may be present at levels such as 0.3% to 25%, 0.3% to 20%, 1% to 25%, 0.5% to 15%, 0.5% to 10%, 0.2% to 6%, 0.5% to 5%, or 0.5% to 3%, or less than 3%, less than 2%, less than 1%, or from 0.1% to 2%.

As used herein, “water-swellale polymers” are polymers than can swell substantially in water and therefore can contribute to a thickening effect of an aqueous solution, particularly at a pH below about 5 such as from 2.5 to 5. They may be water soluble but need not be. Hydrogel formers are examples of such polymers. Water swellable polymers may include known superabsorbent materials such as polyacrylates and co-polymers or cross-polymers thereof and various cellulose derivatives such as hydroxymethyl cellulose or other water soluble cellulosic derivatives, Water-swellable polyurethanes such as those described in WO2004029125A1, etc., may be considered.

As used herein, “silicone compounds” include the numerous silicone derivatives used in cosmetic chemistry and in other personal care applications, often for the smooth textural properties they may impart to an emulsion such as a cream or a solid. Such compounds may include siloxanes such as cyclopentasiloxane, dimethicones, alkyl dimethicones, silesequioxanes including powders such as polymethylsilsequioxane, various liquid silicones or silicone oils such as polydimethylsiloxane (dimethicone), polymethylphenylsiloxane (diphenyldimethicone), phenyltrimethicone, diphenylsiloxyphenyltrimethicone, amino-modified silicones, epoxy-modified silicones, carboxy-modified silicones, polyether-modified silicones, alkyl-modified silicones, and so forth, further including the silicone compounds mentioned in US Patent Application No. 20070196309A1, “Linear silicone resins in personal care applications,” published Aug. 23, 2007 by C. Tarletsky et al., etc.

One challenge in silicone usage, particularly with dimethicones and dimethicone derivatives, is the challenge of getting a stable emulsion with silicone compounds blended with oil or water or especially oil and water mixtures, particularly for silicone levels about roughly 5% of the composition and perhaps especially at low pH. Some of our initial trials revealed that silicone levels above 5% or so could lead to syneresis (sweating) and other stability issues, but it is believed that the inventive use of a suitable thickener combined with the mandelic acid or other alpha-hydroxy acid (e.g., lactic acid, glycolic acid, etc.) can lead to enhanced stability for the mixture and better integration of elevated silicone levels. In some tests, we have successfully integrated over 30% silicone compounds into a mix also comprising lipids and an aqueous phase, which is believed to be an unusual achievement. Thus, in some embodiments, there is provided a stable composition comprising lipids (esters and waxes) at 5% to 70% by weight, significant silicone compounds (5% to 40% by weight), and a thickened aqueous phase carrying enough of one or more acids such as NAC or an alpha-hydroxy acid that is solid in its pure form at room temperature such as mandelic acid to provide at least 1′)/0 or at least 2% of the acid in the stick but less than 20% water in the stick. Likewise, in some embodiments, a method is provided for making such a stick by combining the lipids and silicones in a molten phase, adding at least one of an emulsifier or gelling agent, blending in a thickened aqueous phase comprising the alpha-hydroxy acid to form a low pH molten mass, and optionally adding a powder prior to pouring the molten mass to form a deodorant stick having an effective pH from 2 to 6, or from 2 to 5.5, or from 2.5 to 5.

As used herein, a “semi-solid” refers to a combination of solid and liquid materials or a composite material with multiple phases or discrete components which does not readily flow under the force of gravity when a unit such as a 5-cm cube of the material rests on a flat surface at 20° C., but which can deform and flow under shear. When measured at a shear rate of 0.5 sec⁻¹, the viscosity may be at least 15,000 centistokes, such as from 15,000 to 10,000,000 centistokes, from 30,000 to 10,000,000 centistokes, from 50,000 to 5,000,000 centistokes, or from 80,000 to 5,000,000 centistokes. Commercial deodorant sticks, whether based on waxy material, aqueous gels, or silicone compounds, are commonly semi-solids. Viscosity can be measured with a Brookfield viscometer.

As used herein, “emulsifying wax” refers to waxy materials that promote emulsification of an aqueous or polar phase with an oily, non-polar phase. Emulsifying waxes generally contain compounds derived from fatty acids such as fatty alcohols, esters, and other materials such a Polysorbate 60 or other emulsifiers. Emulsifying waxes may be plant derived, such as derivatives of palm oil, soy oil or olive oil. Examples include the combination of cetearyl alcohol and glyceryl stearate, such as Ritamulse SCG (sometimes known as Emulsimulse) from Rita Corp. (Crystal Lake, Ill.), which is a combination of glyceryl stearate, cetearyl alcohol, and sodium stearoyl lactylate; Emulsifying Wax NF (cetostearyl alcohol and polysorbate 60), and Polawax (cetearyl alcohol, PEG-150 stearate, polysorbate 60, and steareth-20). A useful plant-derived example is Milliard® All Natural Emulsifying Wax (Milliard Brands, Lakewood, N.J.) said to be derived from palm oil, with the INCI name of cetostearyl alcohol and polysorbate 80. Another natural emulsifying wax derived from olive oil is the combination of cetearyl olivate and sorbitan olivate, marked at Olivem 1000@ by Hallstar Beauty (Chicago, Ill.). Emulsifying waxes are generally taught to be used at a level of 2 to 5% of the mass of the emulsion being made (e.g., the combination of the oil and aqueous phases). In some cases, we have found higher levels are useful, as 5% or greater, 6% to 23%, 7% to 17%, or 7% to 15%, and 8% to 14% of the combined mass of the oil and solvent phase prior to the addition of further solids such as starch.

As used herein, the “waxy phase” consists of the non-aqueous or non-polar materials that are combined as part of the process for forming the deodorant stick described herein. It may comprise waxes, oils, fatty acids, and related oleophilic materials. Thus, the waxy phase may comprise:
waxes such as beeswax, white beeswax, candelilla wax, carnauba wax, microcrystalline wax, paraffin wax, hydrogenated castor oil, rice bran wax, berry wax, myrica fruit wax, laurel wax, castor wax (hydrogenated castor oil), sunflower wax, rose wax, orange wax, momosa wax, jasmine wax, polyethylene wax and other synthetic waxes such as Synkos waxes (Kostor Keunen, Waterford, Conn.), and so forth;
alcohols such as stearyl alcohol, behenyl alcohol, cetyl alcohol, cetearyl alcohol, isostearyl alcohol, lauryl alcohol, oleyl alcohol, caprylic alcohol, myristyl alcohol, polyols such as glycerin, glycols such as pentylene glycol, and the like;
fatty esters such as isopropyl myristate, isopropyl palmitate: Used in cosmetics both as a thickening agent and emollient, glyceryl stearate, tridecyl trimellitate, jojoba esters, pentaerythrityl tetraisostearate and other pentaerythrityl tetraesters, lauryl laurate, cetyl esters, PEG stearate, etc.;
oils such as Helianthus annus (sunflower) seed oil, soybean oil, almond oil, walnut oil, coconut oil, caprylic/capric triglicerides, MCT oil, jojoba oil, castor oil, avocado oil, etc.;
fatty acids such as stearic acid, palmitic acid, lauric acid, oleic acid, myristic acid, palmitoleic acid, etc.;
butters such as shea butter, Mangifera indica (mango) seed butter, etc.; and
soaps (metal salts of fatty acids) such as zinc stearate, magnesium stearate, magnesium myristate, sodium oleate, etc.

As used herein, “slip modifiers” are texturizing agents that create an increased sense of slip when a solidified product slides against human skin. Such agents may include, without limitation, caprylyl dimethicone, polymethylsilsesquioxane, aluminum silicate, silica, alumina, talc powder, boron nitride, microcrystalline cellulose, kaolin powder, magnesium aluminum silicate, magnesium silicate, magnesium trisilicate, oleamide, polytetrafluoroethylene, veegum, zinc laurate, zinc myristate, zinc palmitate, zinc resinate, and zinc stearate.

### BRIEF DESCRIPTION OF THE DRAWINGS

FIG. 1 depicts a stick of deodorant in a dispenser according to certain embodiments of the present invention.

### DETAILED DESCRIPTION

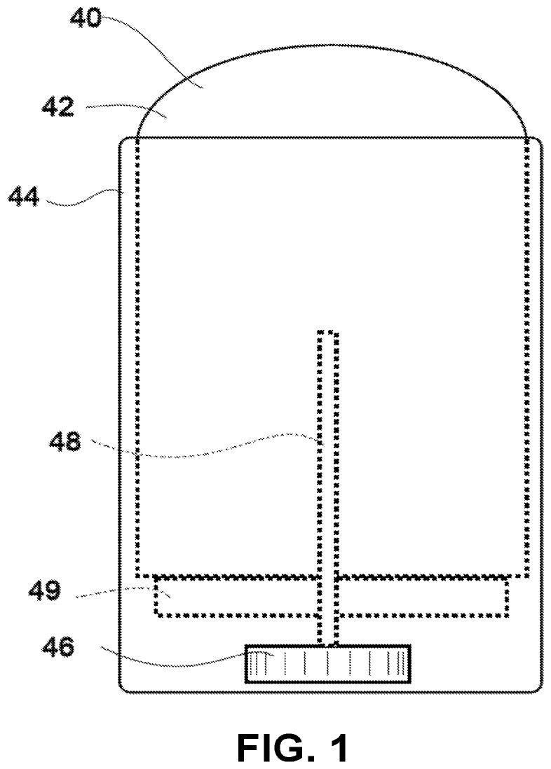

FIG. 1 depicts a stick dispenser 40 comprising a stick of a deodorant or antiperspirant composition 42 contained within the walls of a container 44 having a turnable knob 46 attached to a threaded spindle 48 that drives the motion of an associated platform 49 on which the stick of a deodorant composition 42 rests. Thus, by turning the knob 46, the amount of the stick of a deodorant composition 42 rising above the walls of a container 44 can be adjusted. A cap for the container 44 is not shown. Such dispensers may be filled by pouring a slurry or melt into the dispenser and allowing the slurry to harden, or may be formed by packing solid or semi-solid material into the dispenser and then compressing it and/or heating it to form a suitable deodorant stick.

### Further Details

Many other ingredients may be present in the acidic sticks described herein, including the Vitamin C compounds and other ingredients listed in U.S. RE38623, “Stabilization of ascorbic acid, ascorbic acid derivatives and/or extracts containing ascorbic acid for topical use,” issued Oct. 12, 2004 to S. Hernandez et al.; US Patent Application No. US20070172436A1, “Nonaqueous ascorbic acid compositions and methods for preparing same,” published Jan. 23, 2006 by J. Zhang; and US Patent Application No. 20180071205, “Stable Vitamin C System,” published Mar. 15, 2018 by J. Disalvo.

In some embodiments, urea may also be present as a solubility enhancer, but in other embodiments, the product is substantially free of urea as well as urea derivatives such as mono-, di-, tri-, and tetra-substituted urea compounds, and may also be substantially free of other diamides or of other amides in general, particularly those that are not affirmatively described herein as ingredient candidates.

Products described herein may also comprise various derivatives from fungi and lichens including usnic acid (e.g., from 0.01% to 1.5%), but may also be substantially free of usnic acid and/or substantially free of fungal and/or lichen extracts or derivatives.

Preservatives may be added if desired, or the product may be substantially preservative free. Preservatives may include phenoxyethanol, benzoic acid or salts thereof such as sodium benzoate, tris(N-hydroxyethyl) hexahydrotriazine, etc.

### Examples

The ingredients mentioned in the Examples below were drawn from the following, unless otherwise specified:

The ingredients mentioned in the examples below were drawn from the following:
almond oil: Formulator Sample Shop, Iron Station, N.C.
aluminum chlorohydrate powder: Formulator Sample Shop, Iron Station, N.C.
arrowroot starch: Bob's Red Mill, Milwaukie, Oreg.
beeswax: Sky Organics, Delray Beach, FLUORESCENT
behenyl alcohol: Formulator Sample Shop, Iron Station, N.C.
Bentone Gel GTCC V (a gel-making compound comprising pre-dispersed modified hectorite clay in an oleophilic base; INCI name is caprylicc triglyceride and stearalkonium hectorite and propylene carbonate): Elementis, East Windsor, N.J.
C26-28 alkyl dimethicone (Botanisil AD-1): DDChemco, Chatsworth, Calif.
cacao butter: Cacao Butter Wafers, Terrasoul Foods, Fort Worth, Tex.
caffeine: 200 mg capsules, Bulk Supplements, Henderson, Nev.
candelilla wax: TKB Trading, Oakland, Calif.
caprylyl dimethicone (Botanisil CPM-10): DDChemco, Chatsworth, Calif.
caprylic capric triglycerides MCT: Formulator Sample Shop, Iron Station, N.C.
castor wax (hydrogenated castor oil): Health and Beauty Oils Center, Ebay.com
cetyl alcohol: Formulator Sample Shop, Iron Station, N.C.
cocoamidopropyl hydroxysultaine: Snoqualmie, Wash. (a liquid surfactant used in some baby shampoos due to its gentleness)
coconut oil: LouAna Coconut Oil, Ventura Foods, Brea, Calif. corn starch: Hodgson Mill, Effingham, Ill.
CreamMaker® Behenyl: Making Cosmetics, Snoqualmie, Wash.
cyclopentasiloxane: TKB Trading, Oakland, Calif.
dimethicone and dimethicone/vinyl dimethicone copolymer (Lotioncrafter EL61): Lotioncrafter, Eastsound, Wash.
DM6 dimethicone (dimethicone with a viscosity of 6 cst): Lotioncrafter, Eastsound, Wash.
DM350 dimethicone (dimethicone with a viscosity of 350 cst): Lotioncrafter, Eastsound, Wash.
diphenyl siloxy phenyltrimethicone (Lotioncrafter LC1550): Lotioncrafter, Eastsound, Wash.
ECOMulse™ (an emulsifier made from glyceryl stearate, cetostearyl alcohol, and sodium stearoyl lactylate): Lotioncrafter, Eastsound, Wash.
Ecosil (Fision® EcoSil from Tri-K, a naturally derived blend of hydrogenated ethylhexyl olivate and hydrogenated olive oil unsaponifiables): Formulator Sample Shop, Iron Station, N.C.
emulsifying wax: Milliard® Emulsifying Wax (cetostearyl alcohol and polysorbate 60), Milliard Brands, Lakewood, N.J. (unless otherwise specified, “emulsifying wax” refers to this product)
ethylhexyl palmiate: Making Cosmetics, Snoqualmie, Wash.
FSS Sensolv (isoamyl laurate): Formulator Sample Shop, Iron Station, N.C.
hydroxpropylcocoate PEG-8 dimethicone (Botanisil TE-3): DDChemco, Chatsworth, Calif.
cocoamidopropyl hydroxysultaine: Making Cosmetics
Kostol PGP (an emulsifying wax comprising polyglyceryl-3 stearate, beheneth-5): Koster Keunen, Watertown, Conn.
laponite powder: Laponite XL21, BYK USA, Inc., Gonzales, Tex.
lauryl laurate: TKB Trading, Oakland, Calif.
lip stick base: TKB Trading, Oakland, Calif. (a mixture of castor seed oil, cetyl stearyl alcohol, olive fruit oil, beeswax, hydrogenated castor oil, glycine soybean lipids, lauryl laurate, carnauba wax, candelilla wax)
magnesium myristate: TKB Trading, Oakland, Calif.
magnesium stearate: MarkNature, location unknown.
mandelic acid powder: Pure Health Botanicals, St. Charles, Ill.
panthenol (vitamin B5): L'eternal World LLC, Aurora, Ohio
palmitic acid 98%, Acme-Hardesty, Blue Bell, Pa.
PEG-8 beeswax (esterification of the free fatty acids of beeswax with polyethylene glycol): Koster Keunen, Watertown, Conn.
polyethylene wax: Making Cosmetics, Snoqualmie, Wash.
polymethylsilsesquioxane (Botanisl SP-360): DDChemco, Chatsworth, Calif.
1-3 propanediol: Formulator Sample Shop, Iron Station, N.C.
propylene glycol: Earthborn Elements, American Fork, Utah•shea butter (unrefined): TKB Trading, Oakland, Calif.
Softisan: FSS Softisan 378, Formulator Sample Shop, Iron Station, N.C. (this material is a lanolin-like material made from a blend of triglycerides based on saturated even-numbered, unbranched natural fatty acids of vegetable origin)
stearyl alcohol: Alcohol 1989 N F Pastilles, Acme-Hardesty, Blue Bell, Pa.
sunflower wax: Making Cosmetics, Snoqualmie, Wash.
Synkos 2050 wax: Koster Keunen, Waterford, Conn.
tapioca starch: Erawan Marketing Co., Bangkok, Thailand
TKB gelmaker CC: TKB Trading, Oakland, Calif. (this material is a blend of dicaprylyl carbonate, stearalkonium hectorite and propylene carbonate, and is used to create gels)
water is distilled water unless otherwise specified
xanthan gum: Carrington Farms, Closter, N.J.
zinc stearate: TKB Trading, Oakland, Calif.

Unless otherwise stated, compositions described below were made in a double boiler constructed by using a muffin baking pan with 4×3 muffin wells and external dimensions at the rim of about 13.9 inches×10.6 inches and a well depth of about 1.2 inches and a well diameter of about 2.75 inches, purchased at Walmart in Appleton, Wis. The muffin pan could fit snugly within a large Wilton® baking pan having internal dimensions near the top of the slightly tapered pan of about 14.4 inches×10.8 inches×2 inches, purchased at the same store. During formulation work, the baking pan was placed on a gas stove covering two burners, then filled with water to a depth that allowed the muffin pan to float. The burners could then be turned on to bring the water to a suitable temperature for melting wax and other components in one or more of the wells. The muffin pan came with a detached well that had not been welded to the main pan, a manufacturing defect that provided additional convenience since the loose well could serve as a convenient weighing pan and could, when needed, be placed directly inside one of the other wells to melt and mix ingredients, after which the contents could be weighed if desired to see, for example, how much moisture may have evaporated. The open well port also allowed an easy way to add water conveniently or to preheat utensils such as whisks or spoons.

Illustrative runs made with significant antiperspirant content are shown in runs P1-P4. Ingredients added to the oil-silicone phase are shown in Table 1A, including the “water phase add (addition),” which states how much of the acid paste for each run was combined with the oil-silicone phase. The respective acid paste composition is shown in Table 1B. The overall composition of the final stick is shown in Table 1C.

TABLE 1A
Several antiperspirant runs with oil-silicone phases.

| | Run: | | | |
|---|---|---|---|---|
| Oil phase + silicone phase | P1 | P2 | P3 | P4 |
| aluminum chlorohydrate | 5.5 | 4.2 | 5.03 | 3.994 |
| arrowroot starch | 2 | 3.56 | 3.56 | 4.15 |
| behenyl alcohol | | | 1.03 | |
| Bentone Gel GTCC V | 1.608 | 1.77 | | |
| C26-28 alkyl dimethicone | 1.302 | 1.56 | 1.06 | 1.13 |
| cacao butter | | | | 0.45 |
| caprylyl dimethicone | 1.949 | 0.12 | 1.55 | 0.90 |
| CreamMaker ® Behenyl | | | | 0.69 |
| DM6 dimethicone | 6.03 | 6.63 | 3.93 | 2.28 |
| DM350 dimethicone | 0.85 | 0.94 | 1.72 | 1.00 |
| Ecosil | | | 1.446 | |
| Gelnnaker CC | | | 1.15 | 0.67 |
| isoamyl laurate (Sensolv) | 4.083 | 4.326 | 1.737 | 2.489 |
| Jojoba oil | | | 1.526 | 0.455 |
| Kostol PGP | 0.216 | 0.409 | 0.518 | |
| panthenol | 0.251 | | 0.15 | 0.217 |
| polyethylene wax | | | | 2.143 |
| polymethylsilsesquioxane | 2.896 | 2.547 | 2.12 | 2.54 |
| (SP-360) | | | | |
| squalane | | | 0.26 | 0.15 |
| stearyl alcohol | 2.5 | 1.3 | 3.23 | 2.076 |
| Synkos 2050 wax | 8 | 7.59 | 5.261 | 3.686 |
| Acid paste addition: | 7 | 3.95 | 7.17 | 6.05 |
| Water correction (est. evap. | 0 | | 0.1 | 0.05 |
| loss) | | | | |
| Total mass (g): | 44.19 | 38.91 | 42.34 | 35.02 |

TABLE 1B
Acid paste compositions corresponding to the “acid
paste addition” listed in Table 1A.

| Water Phase | | | | |
|---|---|---|---|---|
| Composition | AP21 | AP29 | AP30 | AP31 |
| Water | | 10.25 | 2.46 | 0.30 |
| Tapioca Starch | | | 0.00 | |
| Mandelic Acid | 8.89 | 6.90 | 1.65 | 2.80 |
| NAC | | 3.34 | 0.80 | 2.00 |
| Glycerin | | | 0.00 | |
| Propane(1,3)diol | 20.28 | 4.14 | 0.99 | 5.30 |
| Propylene glycol | | | 0.00 | |
| Caffeine | 0.00 | | 0.00 | |
| NaOH | 0.00 | 0.26 | 0.06 | |
| Alum. Chlorohydrate | | 3.90 | 0.93 | |
| Allantoin | | | 0.37 | |
| GLDA | | | 0.13 | |
| Cocoamido Hyd. | | | 1.59 | 0.47 |
| Sultaine | | | | |
| Initial mass: | 29.18 | 28.79 | 8.99 | 10.87 |

TABLE 1C
Overall percentage of ingredients in final sticks.

| Summary of Key | | | | |
|---|---|---|---|---|
| Components: | P1 | P2 | P3 | P4 |
| Aluminum chlorohydrate %: | 12.4% | 12.2 | 13.6 | 11.4% |
| Caffeine %: | 0.00% | 0.00 | 0.00 | 0.00% |
| Mandelic acid %: | 4.83% | 2.43 | 3.12 | 4.45% |
| Silicone compounds %: | 29.5% | 30.3 | 24.5 | 22.4% |
| Synthetitc wax °/0: | 18.1% | 19.5 | 12.4 | 16.6% |
| Starch %: | 4.53% | 9.15 | 8.41 | 11.8% |
| Water %: | 0.00% | 3.61 | 4.39 | 0.33% |
| Glycerin %: | 0.00% | 0.00 | 0.00 | 0.00% |
| Esters (with triglycerides) %: | 9.2% | 11.1 | 11.7 | 10.1% |
| Fatty alcohols & acids %: | 5.66% | 3.34 | 10.1 | 5.9% |
| NAC: | 0.00% | 1.18 | 1.51 | 3.18% |
| Diol/glycol %: | 11.0% | 1.46 | 1.87 | 8.42% |
| NaOH: | 0.00% | 0.09 | 0.12 | 0.00% |
| Gelling agents, emulsifiers: | 4.13% | 5.60 | 3.94 | 3.88% |
| Panthenol: | 0.57% | 0.00 | 0.35 | 0.62% |
| Allantoin | | 0.00 | 0.70 | 0.00% |
| GLDA | | 0.00 | 0.24 | 0.00% |
| Hydroxysultaine | | | 3.00 | 0.75% |
| Total: | 100% | 100% | 100% | 100% |
| pH: | 3.9 | 3.8 | 3.5 | 3.7 |

After pouring into round deodorant molds, it was observed that the solid sticks had a good feel and a uniform texture. Sticks AP1 and AP2 were tested on human underarm skin without irritation and no sense of grittiness. The hardness of stick AP2 was measured using an AMS 59032 E-280 Pocket Penetrometer, measured by increasing the applied pressure slowly as the tip engaged the wax until there was a sudden breakthrough and then reading the peak pressure indicated by a movable rubber ring. The units are in kg/sq·cm or tons/sq. foot (1 kg/sq·cm=1.02 tons/sq. foot). A hardness of 1.25 was recorded. Somewhat lower hardness may be desirable in some embodiments.

This approach to acid stick production is built upon inventive work seeking to overcome the basic challenges of producing an acidic solid stick. The related experimental for that initial phase of developing the inventive product as claimed herein is shown in the examples below, illustrating some of the scope of the novel approach to creating acidic sticks.

In many runs prior to run 100, separate oil and silicone phases were prepared and heated, and after heating to 70° C. to 85° C., depending on the particular mix of compounds, were then combined in a single well in the double boiler system and mixed by hand with a whisk or whisk and spoon, together or in succession. Then the acid paste/water phase mixture was added and blended using a whisk or combination of spoon and whisk, followed by addition of starch and possible other powdered materials such as polydimethylsilsequioxane. At that point final ingredients could be added such as essential oils and/or caprylyl dimethicone, though in later embodiments (after run 106) caprylyl dimethicone was blended into the silicone and oil phase prior to mixing with the water phase. After blending in of the starch and other powdered ingredients and the final ingredients, if any, the hot mixture was immediately poured into a deodorant mold, using various molds such as repurposed commercial deodorant containers, 15 ml oval shaped deodorant molds, 2.2 ounce round plastic molds, and clear acrylic cylindrical Juvitus® brand (Culver City, Calif.) 1-ounce molds.

Starting with run 106, all silicone compounds including caprylyl dimethicone, if present, were combined and heated with the oil phase. Starting with run 98, the Bentone gel and dimethicones or other silicone liquids were combined in a large batch, large enough for over 3 runs, and then blended with an immersion mixer before adding to the oil phase and other ingredients, and the mixture was then heated and stirred/whisked together prior to the addition of a heated acid paste, followed by arrowroot starch and polymiethylsilsequioxane powder, when present.

In Tables 2A through 4C below, ingredients blended into the combined oil and silicone phase are shown in Tables 2A, 3A, and 4A, including oils, waxes, and esters including triglicerides, emulsifiers, silicone compounds, and powders that were blended into the mix. The water phase ingredients (also sometimes called the acid paste) are show in Tables 2B, 3B, and 4B, and the amount of the respective water phase/acid paste blended with the oil and silicone phase is listed as the entry for “Water phase add. (addition)” toward the end of each of Tables 2A, 3A, and 4A, which each have slight differences in the collective group of ingredients used. With the combination of the oil and silicone phase, the water phase, and other final ingredients (arrowroot starch, optionally caprylyl dimethicone, optionally polymethylsilsequioxane and fragrances in some early runs), the calculated net composition by ingredient categories is shown in each of Tables 2C, 3C, and 4C. In some cases, as estimate for water loss during mixing is provided which is based on measured mass losses for water during its blending into a hot oil phase, based on rough measurements made as a heated mass was blended over time, using the removable well as an easy-to-weigh container in some experiments, and considering the duration of time at elevated temperature prior to pouring and cooling. The estimated water loss during processing is entered in Tables 2A, 3A, and 4A below the “Water Phase Addition” numbers.

Not all runs are shown, sometimes because they involved peripheral experimental work outside the scope of this disclosure, or occasionally involves experimental mistakes (e.g., adding excessive starch) or other problems. Many early runs focused on simply demonstrating the possibility of making a stable and non-gritty deodorant at all with high mandelic acid content and employed combinations with existing commercial products that often resulted in problems with texture, stability, etc. For example, combinations of the acid paste with existing deodorants high in alkaline materials such as sodium bicarbonate or magnesium hydroxide resulted in frothing, instability, or other setbacks or could not reach desired pH levels without excessive and wasteful additional mandelic acid. Some of these are reported but not all.

TABLE 2A
Series of runs with oil, silicone, and water.

| | Run: | | | | | | | | | | |
|---|---|---|---|---|---|---|---|---|---|---|---|
| Oil + silicone phase | 74 | 75 | 76 | 77 | 78 | 79 | 80 | 81 | 83 | 84 | 85 |
| Arrowroot starch | | | 2.82 | 2.71 | 2.81 | 8.68 | 3 | 5.3 | | 7.38 | 7.31 |
| Beeswax | 1.834 | | | | | | | | | | |
| Behenyl alcohol | | | | 1.281 | | 4.078 | 2.4 | | | | |
| Behenyl CreamMaker | | | | 0.523 | | | | | 0.51 | 0.607 | 0.60 [symbol] |
| Bentone Gel GTCC V | 3.011 | 2.51 | 3.041 | 2.01 | 0.8 | 2.05 | 2.2 | 2.45 | 1.123 | 1.676 | 1.67 [symbol] |
| C26-28 alkyl DM | 1.21 | 4.907 | 4.628 | 0.429 | | 1.093 | 0.62 | 3.2 | 1.572 | 2.514 | 2.51 [symbol] |
| Cacao butter | 0.929 | 4.225 | 7.74 | | | | | | | | |
| Caffeine (oil/sil. [symbol] | | | | 0.2 | | | 0.2 | 0.2 | 0.2 | 0.2 | 0.1 |
| Candelilla wax | 1.021 | 1.206 | 0.743 | 0.538 | | 0.535 | 1.754 | 0.37 | | 0.505 | 0.50 [symbol] |
| Caprylic/capric trigly. | | | | 5.128 | | | | | | | |
| Caprylyl dimethicone | | | | | | | | | 0.3 | | |
| Castor wax | | | | | | | | | | 0.723 | 0.73 |
| Cetyl alcohol | | | | 2.17 | 3.321 | 1.623 | | | | | |
| DM-v-DM copolymer | | | | | | | | | | | |
| DM6 dimethicone | | | | 0.241 | 0.376 | 0.25 | | 0.355 | 1.172 | 0.262 | |
| DM350 dimethicone | | 20. [symbol] | 0.37 | 0.638 | 0.652 | 0.232 | 0.917 | 0.986 | 0.67 | 0.495 | 0.49 [symbol] |
| DM4200 dimethicone | | | | | | | | 0.441 | | | |
| Diphenyl siloxy PTM | | | | 0.205 | 0.372 | 0.353 | 0.54 | 0.603 | 0.154 | 0.27 | 0.27 |
| Ecosil | | | 1.777 | | | 16.21 | 10.9 | 12.84 | | | |
| Ethylhexyl palmiate | | | 1.64 | 0.87 | | 0.223 | 0.669 | | 0.402 | | |
| Glycerin (oil phase) | | | 1.26 | 0.73 | 0.635 | 0.787 | 0.787 | 1.569 | | | |
| Hemisqualane | 2.99 | | | | | | | | | | |
| Hydroxpr.-cocoate | 0.841 | | | | | | | | | | |
| PEG-8 DM | | | | | | | | | | | |
| Isoannyl laurate | | | | | 4.958 | | 2.61 | | 4.26 | 7.07 | 7.08 |
| Kostol PGP | | | | | | 0.26 | 0.403 | 0.505 | 0.105 | 0.176 | 0.17 [symbol] |
| Lauryl laurate | | | | 4.49 | 2.3 | 7.4 | 3.41 | 4.028 | | | |
| Magnesium stearate | | | | | | | | | | 0.492 | |
| Panthenol | | | | | 0.147 | 0.285 | 0.352 | 0.3 | 0.151 | 0.253 | 0.25 [symbol] |
| Polymethylsilsesquio | 3.011 | | 1.00 | | | | 2.93 | | | 1.85 | 1.59 |
| Silica dimethyl silylyl | | | | 0.78 | | 1.303 | 0.365 | | | | 0.48 |
| Squalane | | 0.904 | 0.83 | 0.53 | 0.383 | | | 0.586 | 0.809 | 0.399 | 1.04 [symbol] |
| Stearyl alcohol | | | 1.69 | 1.008 | | 2.778 | 4.068 | 8 | 3.033 | 5.005 | 5.00 [symbol] |
| Water phase add.: | 9.2 | 3.07 | 2.4 | 2.465 | 2.433 | 3.51 | 3 | 4 | 2.63 | 4.26 | 4.49 |
| Water loss (est.) | 0.2 | 0.1 | | 0.1 | 0.1 | 0.1 | | | | 0.1 | 0.1 |
| Total mass (g): | 23.85 | 37.41 | 29.94 | 26.85 | 19.09 | 51.55 | 41.13 | 45.73 | 17.09 | 34.04 | 34.2 |

[symbol]

indicates data missing or illegible when filed

Not listed are fragrances in some cases. For example, run 74 had 3 drops (0.076 g) of elemi essential oil added before pouring.

In run 74, the polymethylsilsequioxane powder (3.011 g) was added to a first oil-silicone phase with 1.83 g beeswax, 1.021 g candelilla wax, 0.929 g cacao butter, and 1.21 g C26-28 alkyl dimethicone, while an oil-gel phase was made from 0.841 g of hydroxypropyl-PEG-8 dimethicone, 3.011 g of Bentone gel, 2.999 g hemisqualane, and essential oil. Once heated and blended separately, the two were combined at 80° C. and then 9.2 g of Acid Paste 10 was gradually blended in with a whisk. This occurred in the removable well allowing weighing of the unit before, during, and after the blending process. Acid Paste 10 comprised glycerin as the thickener with about 15% glycerin and about 15% mandelic acid present in the aqueous phase. The final product had 24% water, 6% each of glycerin and mandelic acid, over 21% silicones, etc. The resulting product was too soft and not a viable candidate for a stick, possibly because of too high a water level for a silicone+oil+water+acid+thickener formulation. In this case, in retrospect it is proposed that a water level less than 23%, less than 20%, less than 18%, or less than 16%, 15%, 14%, 13%, 12%, 11%, 10% or 9%, such as from 2% to 20° A, 3% to 20%, 4% to 18%, 5% to 23%, 1% to 12%, etc., may have been helpful in providing a more suitable, harder composition.

Several different acid pastes were used for runs 75-85, as shown in Table 2B.

TABLE 2B
Water phase compositions used for “water phase”
addition listed in Table 2A for the runs listed.

Water Phase ID

| | | | |
|---|---|---|---|
| AP10 | AP10A | AP 11 | AP 12 |

Used in runs:

| Composition: | 74 | 75 | 76-81 | 82-85 |
|---|---|---|---|---|
| Water | 21.7 | 24.73 | 31 | 10.90 |
| Tapioca Starch | 0 | 0.44 | 1.35 | 0.47 |
| Mandelic Acid | 5.108 | 4.06 | 1.878 | 0.66 |
| NAC | 0 | 0.62 | 1.901 | 0.67 |
| Glycerin | 5.04 | 3.40 | 0 | 2.50 |
| Propane(1,3)diol | 0 | 0 | 0 | 1.10 |
| Propylene glycol | 0 | 0 | 0 | 1.52 |
| Caffeine | 0 | 0 | 0 | 0.00 |
| NaOH | 1.29 | 0.87 | 0.00 | 0.00 |
| Initial mass: | 33.14 | 34.11 | 36.13 | 17.82 |

TABLE 2C
Overall percentage of ingredients in final sticks.

| | Run: | | | | | | | | | | |
|---|---|---|---|---|---|---|---|---|---|---|---|
| Oil + silicone phase | 74 | 75 | 76 | 77 | 78 | 79 | 80 | 81 | 83 | 84 | 85 |
| Caffeine %: | 0.00 [symbol] | 0.00 [symbol] | 0.00 [symbol] | 0.74 [symbol] | 0.00 [symbol] | 0.00 [symbol] | 0.49 [symbol] | 0.44 [symbol] | 1.10 [symbol] | 0.57 [symbol] | 0.29 [symbol] |
| Mandelic acid %: | 5.95 [symbol] | 0.98 [symbol] | 0.42 [symbol] | 0.48 [symbol] | 0.60 [symbol] | 0.35 [symbol] | 0.38 [symbol] | 0.45 [symbol] | 0.54 [symbol] | 0.45 [symbol] | 0.47 [symbol] |
| Silicone %: | 21.2 [symbol] | 68.4 [symbol] | 20.0 [symbol] | 8.54 [symbol] | 7.33 [symbol] | 6.27 [symbol] | 13.1 [symbol] | 12.2 [symbol] | 27.3 [symbol] | 17.8 [symbol] | 17.6 [symbol] |
| Synthetitc wax %: | 0.00 [symbol] | 0.00 [symbol] | 0.00 [symbol] | 0.00 [symbol] | 0.00 [symbol] | 0.00 [symbol] | 0.00 [symbol] | 0.00 [symbol] | 0.00 [symbol] | 0.00 [symbol] | 0.00 [symbol] |
| Starch %: | 0.00 [symbol] | 0.11 [symbol] | 9.72 [symbol] | 10.4 [symbol] | 15.2 [symbol] | 17.1 [symbol] | 7.57 [symbol] | 11.9 [symbol] | 0.39 [symbol] | 21.5 [symbol] | 21.2 [symbol] |
| Water %: | 24.4 [symbol] | 5.68 [symbol] | 6.88 [symbol] | 7.51 [symbol] | 10.4 [symbol] | 5.65 [symbol] | 6.26 [symbol] | 7.50 [symbol] | 8.84 [symbol] | 7.19 [symbol] | 7.55 [symbol] |
| Glycerin %: | 5.87 [symbol] | 0.82 [symbol] | 4.21 [symbol] | 2.72 [symbol] | 3.33 [symbol] | 1.53 [symbol] | 1.91 [symbol] | 3.43 [symbol] | 2.03 [symbol] | 1.72 [symbol] | 1.80 [symbol] |
| Esters, triglycerides | 28.4 [symbol] | 16.9 [symbol] | 42.5 [symbol] | 43.1 [symbol] | 40.0 [symbol] | 47.3 [symbol] | 47.0 [symbol] | 39.0 [symbol] | 30.1 [symbol] | 25.0 [symbol] | 26.7 [symbol] |
| Fatty alcohols, acids | 0.00 [symbol] | 0.00 [symbol] | 5.64 [symbol] | 16.6 [symbol] | 17.4 [symbol] | 16.5 [symbol] | 15.7 [symbol] | 17.5 [symbol] | 16.7 [symbol] | 14.4 [symbol] | 14.3 [symbol] |
| NAC: | 0.00 | 0.15 [symbol] | 0.42 [symbol] | 0.48 [symbol] | 0.67 [symbol] | 0.36 [symbol] | 0.38 [symbol] | 0.46 [symbol] | 0.54 [symbol] | 0.46 [symbol] | 0.48 [symbol] |
| Diol/glycol %: | 0.00 | 0.00 | 0.00 | 0.00 | 0.00 | 0.00 | 0.00 | 0.00 [symbol] | 2.12 [symbol] | 1.80 [symbol] | 1.88 [symbol] |
| NaOH: | 1.50 | 0.21 [symbol] | 0.00 [symbol] | 0.00 [symbol] | 0.00 [symbol] | 0.00 [symbol] | 0.00 [symbol] | 0.00 [symbol] | 0.00 [symbol] | 0.00 [symbol] | 0.00 [symbol] |
| Gels, emulsifiers: | 12.6 [symbol] | 6.71 [symbol] | 10.2 [symbol] | 9.44 [symbol] | 4.19 [symbol] | 4.48 [symbol] | 6.33 [symbol] | 6.46 [symbol] | 9.56 [symbol] | 7.06 [symbol] | 7.02 [symbol] |
| Panthenol: | 0.00 [symbol] | 0.00 [symbol] | 0.00 [symbol] | 0.00 [symbol] | 0.77 [symbol] | 0.55 [symbol] | 0.86 [symbol] | 0.66 [symbol] | 0.83 [symbol] | 0.73 [symbol] | 0.72 [symbol] |
| Other (MG + ZN | | | | | | | | | | 1.41 [symbol] | |
| Total: | 100 | 100 | 100 | 100 | 100 | 100 | 100 | 100 | 100 | 100 | 100 |
| pH | | 3.3 | 3.2 | | | | | | 3.0 | 4.5 | 3 |

[symbol]

indicates data missing or illegible when filed

Table 3A shows formulations used for runs 93-101, with respective acid pastes shown in Table 3B and final stick compositions by category shown in Table 3C.

TABLE 3A
Series of runs with oil, silicone, and water.

| | Run: | | | | | | | | |
|---|---|---|---|---|---|---|---|---|---|
| Oil phase + silicone | 93 | 94 | 95 | 96 | 97 | 98 | 99 | 100 | 101 |
| Arrowroot starch | 2.54 | 3.8 | 2.51 | 4.31 | 3.71 | 3.82 | 3.82 | 3.71 | 3.55 |
| Bentone Gel GTCC V | 0.92 | 1.06 | 1.06 | 1.68 | 1.82 | 1.78 | 1.59 | 1.65 | 1.65 |
| C26-28 alkyl DM | 2.01 | 1.45 | 1.47 | 1.47 | 2.59 | 2.09 | 2.45 | 2.4 | 1.25 |
| Caprylyl dimethicone | 1.4 | 1.3 | 1.76 | 1.76 | 1.74 | 0 | 3.4 | 1.84 | 1.89 |
| DM6 dimethicone | 1.96 | 2.71 | 2.97 | 4.96 | 5.12 | 5.69 | 6.36 | 6.06 | 6.06 |
| DM350 dimethicone | 0.49 | 0.29 | 0.47 | 0.68 | 0.86 | 0.89 | 0.76 | 0.86 | 0.86 |
| Diphenyl siloxy PMT | 0.94 | | | | | | | | |
| Ecosil | 0.35 | | | | | 4.01 | | | |
| Ethylhexyl palmiate | 1.89 | | | | | | | | |
| lsoannyl laurate | 3.01 | 1.97 | 2.18 | 3.85 | 3.49 | | 4.12 | 4.23 | 4.35 |
| Kostol PGP | 0.28 | | 0.24 | 0.32 | 0.40 | 0.43 | 0.42 | 0.46 | 0.46 |
| Panthenol | 0.24 | 0.27 | 0.11 | 0.18 | 0.20 | 0.20 | 0.27 | 0.25 | 0.25 |
| Polyethylene wax | 3.73 | 5.10 | 4.68 | 7.97 | 8.03 | 7.06 | 7.5 | 8 | 8 |
| Polymethylsilsesquio | 2.1 | 2.01 | 2.01 | 2.95 | 3.50 | 3.79 | 3.79 | 3.05 | 3.05 |
| Stearyl alcohol | 1.70 | 1.93 | 1.89 | 3.22 | 3.30 | 3.33 | 2.5 | 2.5 | 2.5 |
| Synkos 2050 wax | | | | | | | | | |
| Water phase add.: | 5.42 | 5.8 | 4.72 | 9.2 | 7.65 | 7.55 | 7.46 | 7.46 | 7.4 |
| Water loss (est.) | 0.2 | 0.2 | 0.2 | 0.5 | 0.4 | 0.4 | 0.4 | 0.3 | 0.3 |
| Total mass (g): | 28.8 | 27.5 | 25.9 | 42.1 | 42.0 | 40.2 | 44.0 | 42.2 | 41.0 |

TABLE 3B
Water phase compositions used for “water phase”
addition listed in Table 3A for the runs listed.

| | Water Phase Composition | | | | | | |
|---|---|---|---|---|---|---|---|
| | AP14 | AP15 | AP16 | AP17 | AP18 | AP18B | AP19 |
| Used in runs: | 93 | 94 | 95 | 96 | 97 | 98 | 99-101 |
| Water | 50 | 22.2 | 19.79 | 20.27 | 12.27 | 11.50 | 19.40 |
| Tapioca Starch | 3 | 0 | 1.53 | 1.41 | 0.90 | 0.90 | 1.44 |
| Mandelic Acid | 7.94 | 8 | 11.93 | 10.00 | 6.35 | 6.35 | 10.00 |
| NAC | 1.28 | 0 | 0.00 | 0.00 | 0.00 | 0.00 | 0.00 |
| Glycerin | 14 | 3.29 | 2.77 | 0.00 | 0.00 | 0.00 | 0.00 |
| Propane(1,3)diol | 2.75 | 0 | 0.00 | 1.62 | 2.42 | 2.42 | 2.53 |
| Propylene glycol | 2.04 | 0.00 | 0.00 | 0.00 | 0.00 | 0.00 | 0.00 |
| Caffeine | 1.2 | 3.2 | 4.09 | 4.00 | 2.54 | 2.54 | 3.80 |
| NaOH | 0 | 0 | 0.66 | 0.33 | 0.21 | 0.21 | 0.25 |
| Initial mass: | 82.21 | 36.69 | 40.77 | 37.63 | 24.68 | 23.91 | 37.42 |

As Acid Paste 19 was used in several runs with reheating prior to each use, additional water evaporated. The content of 19.40 g of water initially was estimated to be reduced, in effect (in terms of the overall original composition), to 19.10 g for runs 100 and 100. The resulting composition by percentage of the resulting sticks are shown in Table 3C:

TABLE 3C
Overall percentage of ingredients in final sticks.

| Summary of Key | Run: | | | | | | | | |
|---|---|---|---|---|---|---|---|---|---|
| Components: | 93 | 94 | 95 | 96 | 97 | 98 | 99 | 100 | 101 |
| Caffeine %: | 0.27% | 1.84% | 1.83% | 2.33% | 1.87% | 1.99% | 1.72% | 1.81% | 1.85% |
| Mandelic acid %: | 1.82% | 4.60% | 5.34% | 5.81% | 4.68% | 4.98% | 4.53% | 4.77% | 4.87% |
| Silicone compounds | 30.9% | 28.2% | 33.6% | 28.1% | 32.9% | 30.9% | 38.1% | 33.7% | 32.0% |
| Synthetic wax °/0: | 13.0% | 18.6% | 18.1% | 18.9% | 19.1% | 17.5% | 17.0% | 19.0% | 19.5% |
| Starch %: | 9.52% | 13.8% | 10.4% | 11.1% | 9.49% | 10.2% | 9.33% | 9.48% | 9.37% |
| Water %: | 10.8% | 12.0% | 8.08% | 10.6% | 8.10% | 8.03% | 7.87% | 8.39% | 8.56% |
| Glycerin %: | 3.21% | 1.89% | 1.24% | 0.00% | 0.00% | 0.00% | 0.00% | 0.00% | 0.00% |
| Esters, trig lyce rid | 18.2% | 7.17% | 8.43% | 9.15% | 8.31% | 9.97% | 9.35% | 10.0% | 10.6% |
| fatty alcohols & | 5.89% | 7.00% | 7.32% | 7.66% | 7.85% | 8.29% | 5.68% | 5.93% | 6.10% |
| NAC: | 0.29% | 0.00% | 0.00% | 0.00% | 0.00% | 0.00% | 0.00% | 0.00% | 0.00% |
| Diol/glycol °/0: | 1.10% | 0.00% | 0.00% | 0.94% | 1.78% | 1.90% | 1.15% | 1.21% | 1.23% |
| NaOH: | 0.00% | 0.00% | 0.30% | 0.19% | 0.15% | 0.16% | 0.11% | 0.12% | 0.12% |
| Gels, emulsifiers: | 4.17% | 3.86% | 5.02% | 4.76% | 5.29% | 5.50% | 4.58% | 5.01% | 5.15% |
| Panthenol: | 0.83% | 0.99% | 0.43% | 0.43% | 0.48% | 0.51% | 0.61% | 0.59% | 0.61% |
| Total: | 100% | 100% | 100% | 100% | 100% | 100% | 100% | 100% | 100% |
| pH | 2.5 | 2.5-2.9 | 2.9 | 2.9 | 3 | 2.5 | 2.8 | 2.7-2.9 | 3.2 |

Run 93 included some N-acetyl cysteine in the acid paste. Without wishing to be bound by theory, it is proposed and believed that the low pH of N-acetyl cysteine coupled with its potential antimicrobial or anti-biofilm capabilities may be compatible with the mechanisms of mandelic acid in enhancing the skin microbiome and thus may be a particularly useful ingredient for a deodorant, although in high concentrations it can provide a sulfurous odor. The 0.3% concentration in this sample did not lead to obvious sulfurous odors and appeared to be compatible with the formulation. Other earlier runs also showed that even higher concentrations of NAC could be successful and gave positive results in testing on human subjects, though the sulfurous smell of NAC was sometimes noted.

Run 101 was repeated but with 3 different pour temperatures, 78° C., 68° C., and 63° C., with substantially the same quantity poured into identical 2.2-ounce round deodorant molds and cooled to about 72° C. Hardness was measured using the AMS 59032 E-280 Pocket Penetrometer. A hardness of 0.6 was recorded for the pour at 78° C., 0.5 for 68° C., and 0.4 for 63° C.

Table 4A shows formulations used for runs 102-110, with respective acid pastes shown in Table 4B and final stick compositions by category shown in Table 4C.

TABLE 4A
Series of runs with oil, silicone, and water.

| | Run: | | | | | | | |
|---|---|---|---|---|---|---|---|---|
| Oil phase + silicone | 102 | 103 | 104 | 105 | 106 | 108 | 109 | 110 |
| Arrowroot starch | 3.59 | 3.56 | 3.55 | 3.54 | 3.55 | 3.55 | 3.67 | 3.62 |
| Bentone Gel GTCC [symbol] | 1.16 | 1.64 | 1.64 | 1.64 | 1.61 | 1.62 | 1.64 | 1.66 |
| C26-28 alkyl DM | 0.95 | 1.25 | 1.25 | 1.25 | 1.25 | 1.25 | 1.25 | 1.24 |
| Caprylyl [symbol] | 1.89 | 1.89 | 1.89 | 1.84 | 1.932 | 1.88 | 1.96 | 1.94 |
| DM6 dimethicone | 4.25 | 6.00 | 6.00 | 6.01 | 6.05 | 6.10 | 6.17 | 6.27 |
| DM350 dimethicone | 0.60 | 0.86 | 0.86 | 0.86 | 0.86 | 0.86 | 0.88 | 0.90 |
| lsoannyl laurate | 4.3 | 4.35 | 4.38 | 4.38 | 4.37 | 4.37 | 4.36 | 4.54 |
| Kostol PGP | 0.31 | 0.46 | 0.46 | 0.46 | 0.46 | 0.46 | 0 | 0.47 |
| Panthenol | 0.26 | 0.28 | 0.28 | 0.28 | 0.29 | 0.27 | 0.27 | 0.27 |
| Polyethylene wax | 8.03 | 8.04 | 8.03 | | | | | |
| Polymethylsilsesquio | 3.3 | 2.87 | 2.86 | 2.86 | 2.87 | 2.02 | 1.76 | 1.83 |
| Stearyl alcohol | 2.51 | 2.5 | 2.51 | 2.51 | 2.51 | 2.5 | 2.5 | 2.66 |
| Synkos 2050 wax | | | | 8.04 | 8.04 | 8.01 | 8.01 | 8.09 |
| Water phase add.: | 7 | 7.39 | 7.39 | 7.14 | 7 | 7.37 | 7.53 | 6.75 |
| Water loss (est.) | 0.3 | 0.2 | 0.2 | 0.2 | 1 | 0.1 | 0.1 | 0.1 |
| Total mass (g): | 37.8 | 40.9 | 40.9 | 40.6 | 39.8 | 40.2 | 39.9 | 40.1 |

[symbol]

indicates data missing or illegible when filed

TABLE 4B
Water phase compositions used for “water phase”
addition listed in Table 4A for the runs listed.

| | Water Phase Composition | | | | |
|---|---|---|---|---|---|
| | AP19 | AP20 | AP23 | AP24 | AP25 |
| Used in runs: | 102 | 103 | 108 | 109 | 110 |
| Water | 19 | 20 | 19 | 9.7 | 13 |
| Tapioca Starch | 1.44 | 1.43 | 1.44 | 1.56 | 0.68 |
| Mandelic Acid | 10 | 10 | 10 | 11.11 | 10 |
| Propane(1,3)diol | 2.53 | | 2.54 | 11 | 6.5 |
| Propylene glycol | 0 | 2.54 | | | |
| Caffeine | 3.8 | 3.8 | 3.73 | 3.8 | 3.8 |
| NaOH | 0.25 | | 0.325 | 0.345 | 0.347 |
| Initial mass: | 37.0 | 37.8 | 37.0 | 37.5 | 34.3 |

As Acid Paste 20 was used in several runs with reheating prior to each use, additional water evaporated. The content of 20.0 g of water initially was estimated to be reduced by evaporation, in effect, in terms of the overall original composition, to 19.5 g in Run 104, 19.2 g for run 105, and 18.9 g for run 106. The resulting composition by percentage of the resulting sticks are shown in Table 4C:

TABLE 4C
Overall percentage of ingredients in final sticks.

| Summary of Key | Run: | | | | | | | |
|---|---|---|---|---|---|---|---|---|
| Components: | 102 | 103 | 104 | 105 | 106 | 108 | 109 | 110 |
| Caffeine %: | 1.90% | 1.82% | 1.84% | 1.81% | 1.82% | 1.85% | 1.91% | 1.86% |
| Mandelic acid %: | 5.00% | 4.79% | 4.85% | 4.75% | 4.80% | 4.96% | 5.59% | 4.90% |
| Silicone compounds %: | 29.0% | 31.5% | 31.4% | 31.6% | 32.6% | 30.2% | 30.1% | 30.3% |
| Synthetic wax °/0: | 21.2% | 19.7% | 19.6% | 19.8% | 20.2% | 19.9% | 20.1% | 20.2% |
| Starch %: | 10.2% | 9.39% | 9.37% | 9.39% | 9.61% | 9.55% | 9.99% | 9.35% |
| Water %: | 8.70% | 9.08% | 8.97% | 8.64% | 6.55% | 9.17% | 4.63% | 6.12% |
| Glycerin %: | 0.00% | 0.00% | 0.00% | 0.00% | 0.00% | 0.00% | 0.00% | 0.00% |
| Esters, triglycerides | 11.4% | 10.6% | 10.7% | 10.8% | 11.0% | 10.9% | 10.9% | 11.3% |
| Fatty alcohols & acids | 6.63% | 6.11% | 6.14% | 6.17% | 6.30% | 6.22% | 6.27% | 6.63% |
| Diol/glycol %: | 1.26% | 1.22% | 1.23% | 1.21% | 1.22% | 1.26% | 5.53% | 3.18% |
| NaOH: | 0.12% | 0.00% | 0.00% | 0.00% | 0.00% | 0.16% | 0.17% | 0.17% |
| Gelling agents, | 3.88% | 5.14% | 5.13% | 5.18% | 5.21% | 5.18% | 4.11% | 5.31% |
| emulsifiers: | | | | | | | | |
| Panthenol: | 0.68% | 0.69% | 0.68% | 0.70% | 0.72% | 0.67% | 0.68% | 0.67% |
| Total: | 100% | 100% | 100% | 100% | 100% | 100% | 100% | 100% |
| pH: | 2.7 | 2.5 | N/A | N/A | 2.5 | 2.7 | 2.5 | 2.5 |

The majority of the runs shown above resulted in sticks that solidified well with a range of textures suitable for a solid stick. Granules of mandelic acid could not be perceived if they were present. Rather, the sticks were smooth and generally seemed highly uniform. A number of products were tested on human volunteers with excellent performance, both in terms of application and non-irritation, but also in terms of odor control performance. Water levels between 3 and 20% or 5 and 15% appeared to be capable of providing surprisingly high concentrations of mandelic acid without the problem of graininess and irritation of the skin. Based on further experimental work in which large quantities of caffeine and mandelic acid were dissolved in various elevated viscosity fluids such as water and tapioca starch, glycerin, propanediol, and propylene glycol, it was observed that these liquids often can be easily saturated with the dissolved solids at elevated temperature (e.g., 70° C., 75° C., 80° C., 85° C., 90° C., 95° C., 100° C., 110° C. and 120° C.) and then, upon cooling to room temperature, while solids can precipitate, the precipitate tends to be very fine and often difficult for human skin to perceive the existence of a solid phase. While mandelic acid dissolved in water alone can readily give large crystals after cooling, the thickened fluids described herein seemed, without wishing to be bound by theory, to help control crystallization to reduce the formation of large grains, though fine feathery, needlelike particles believed to be caffeine crystals could be seen in a microscope. It is also possible that caffeine preferentially precipitated leaving high levels of mandelic acid in the liquid.

A number of early experimental runs are also described below.

Run 1. Acid Paste 1 was made by combining 13 g of corn starch and 14.26 g of mandelic acid in 100 ml of water and heating while stirring in a frying pan to form a uniform translucent paste of relatively high viscosity. Approximately 17 g of moisture was lost during preparation.

An oil phase was made from 1.96 g of TKB lipstick base, 0.68 g of candelilla wax, 0.52 of shea butter, and 1.62 g of emulsifying wax. After melting, 8.17 g of Acid Paste 1 was blended in. Acid Paste 1 was first heated in a microwave to about 60° C. Then 6.06 g of an aluminum-free commercial deodorant, Ivory® Gentle Aluminum Free Deodorant, Hint of Aloe (2.4 ounce), a product of Procter and Gamble (Cincinnati, Ohio), was melted and stirred into the waxy phase. The ingredients of the Ivory® product are: cyclopentasiloxane, stearyl alcohol, magnesium hydroxide, mineral oil, PPG-14 butyl ether, hydrogenated castor oil, petrolatum, fragrance, cyclodextrin, magnesium carbonate, and behenyl alcohol. The alkaline nature of the Ivory® product required a relatively high amount of mandelic acid to be added to ensure a low pH.

Sodium bicarbonate in the Ivory® product reacted with the mandelic acid and released some carbon dioxide bubbles. After continued stirring and heating, the bubbles died down and the resulting emulsion was cooled. It had a waxy feel but was very firm. Relative to typical commercial deodorant sticks, the viscosity seemed relatively high, but it was tested in underarm use and with multiple passes could deliver what seemed to be a suitable amount. There was no evidence of skin irritation after use. Further, there was no evidence of tangible mandelic acid crystals or phase separation, suggesting such a formula could be used for a deodorant stick. This version comprised both waxy materials, mandelic acid delivered in a thick starchy aqueous paste, and a cyclopentasiloxane base from the Ivory® detergent.

Mandelic acid content is nominally at least 5.0% (more depending on how much water evaporated from the mix).

Run 2. The oil phase was made from 9.00 g of beeswax, 2.5 g of emulsifying wax, and 0.78 g of TKB gelling agent. The heated oil phase was combined with 8.9 g of Acid Paste 1 and, after thorough stirring, was further combined with 9.41 g of molten deodorant taken from Mitchum® Natural Power Bamboo Powder Deodorant for women, manufactured by Revlon (New York, N.Y.). The Mitchum® product comprises fatty ingredients, starch, sodium bircarbonate, and other ingredients. The resulting product after cooling was firm but could be applied to the skin. It had an effective pH of about 4.0 and had at least 3.4% mandelic acid.

Run 3. The oil phase was made from 3.56 g of cetyl alcohol, 3.16 g candelilla wax, 0.17 g zinc stearate, 1.0 g of shea butter, and 1.93 g of lipstick base. After melting, it was combined with 6.0 g of Acid Paste 1, and that was then combined with 10.8 g of molten Ivory® Gentle Aluminum Free Deodorant, Hint of Aloe and cooled. The pliable material was then, while largely solidified, pressed into a deodorant mold from a commercial product. The effective pH was about 6.5 and the mandelic acid content was nominally 2.7%.

Run 21. Acid Paste 4 was made by combining 13 g mandelic acid in 100 ml of water with 8.35 g arrowroot starch and cooking in a frying pan to create a translucent paste. Then 9.0 g almond oil was combined with 4.26 g stearyl alcohol, 2.68 g cacao butter, 0.200 g caffeine and 1.57 g emulsifying wax. After melting, 2.15 g of Acid Paste 4 was blended in, and then, after slight cooling, 3.68 g of arrowroot starch was blended in. A portion of the hot composition was poured into a 15 ml deodorant/lip balm container. The cooled result had a smooth tactile feel but was also on the firm side. The effective pH was 3.0 and the nominal mandelic acid content was 1.0%.

Run 22. 7.36 g of the remaining composition from Example 21 was combined with 0.84 g cacao butter, 0.79 g beeswax, 0.34 g shea butter, and 1.37 g arrowroot starch. The resulting mix had a pH of about 5.5 with about 0.7% mandelic acid. This mix was melted again and 1.25 g of Acid Paste 4 was blended in to further lower the pH. The material did not seem firm enough, so it was remelted and blended with 0.44 g stearyl alcohol and 0.6 g arrowroot starch, but the resulting product was too starchy and did not have the intended strength and consistency.

Run 23. An oil phase was made from 8.9 g caprylic capric triglycerides MCT, 0.91 g cacao butter, 1.00 g shea butter, 2.47 g beeswax, 5.25 g stearyl alcohol, 3.00 g emulsifying wax, 200 mg caffeine, and 1.10 g almond oil. After melting, 4.0 g of Acid Paste 4 was blended in, and then 6.2 g of arrowroot starch was added. The result was too starchy and lacked the strength expected in a waxy composition. A portion of the result, 13.4 g, was melted and combined with 2.1 g stearyl alcohol, 1.15 g almond oil, and 1.35 g candelilla wax. The pH was tested and was on the high side, so the mix was remelted and 4.9 g of additional Acid Paste 4 was blended in. The result had a more acceptable feel and gave an effective pH when wetted of about 3. The nominal mandelic acid content was 3.1%, though it may have been slightly higher due to evaporation of water.

Run 24. The oil phase comprised 4.25 g stearyl alcohol, 1.21 g emulsifying wax, 2.77 g cacao butter, and 3.86 g caprylic capric triglycerides MCT. After melting, 3.4 g of Acid Paste 4 was blended in with about 6 g of arrowroot starch. The effective pH was about 3.0. The result, however, was too starchy. Mandelic acid content was about 1.7%.

Run 25. 13.4 g of Example 24 was melted and combined with 2.1 g of stearyl alcohol, 1.15 g of almond oil, 1.35 g candelilla wax, and then an additional 4.9 g of Acid Paste 4 was added. The result had a firm, fatty texture, was smooth, and could be readily smeared on the skin. The effective pH was about 3.5. The nominal mandelic acid content was 3.4%.

Run 27. Acid Solution 6 was made by combining 17.5 g of mandelic acid with 50 ml of water and heating to dissolve. After heating and stirring, about 5 g of water were lost. This was Acid Solution 6. 3.11 g of tapioca starch were then combined with 3.1 ml of water to form a uniform slurry at about 22° C. Then 25.0 g of Acid Solution 6 was blended into the starch in a beaker, a stirring rod was added and the mixture was placed on a magnetic stirrer hot plate and gradually heated and stirred. As the mixture rose above 50° C., it began to form a thickened paste. This paste is Acid Paste 6.

An oil phase was made from 0.38 g palmitic acid combined with 5.00 g lipstick base, 1.03 g lauryl laurate, 3.00 g coconut oil, 4.60 g stearyl alcohol, 2.43 g cacao butter, and 200 mg caffeine. This was melted in a double boiler. Then 1.26 g of Acid Paste 6 was blended in with a whisk, and as the mixture cooled to about 53° C., 4.5 g of arrowroot starch was blended in. The nominal mandelic acid content was 1.7%.

Run 28. An acidic paste was made from 1.351 g laponite powder and 0.226 g xanthan gum with 25.7 g of Acid Solution 6. This was also stirred and heated on a magnetic stirrer plate until it reach a temperature of 45° C. This was Acid Paste 6XL, 1.00 g of which was stirred into an oil phase formed by heating the following on a double boiler to about 75° C.: 200 mg of caffeine combined with 0.60 g shea butter, 7.00 g lipstick base, 4.50 g stearyl alcohol, 1.13 g beeswax, 1.48 g cacao butter, 0.51 g candelilla wax, and 1.03 g of almond oil. After Acid Paste 6XL was blended with the oil phase using a whisk, 3.00 g of arrowroot starch were blended in and the mixture was spooned into a 15 ml deodorant/lip balm container and cooled. The white, opaque solid had a very firm texture and may have had slightly too much starch for easy dispensing onto the skin, or the laponite and gum combination may have increased the viscosity of the stick relative to some other mixtures with starch. When about 0.1 g was blended with 0.2 g of water and placed on Hydrion® 3.0 to 6.0 pH paper, a pH of about 3.6 was indicated. The nominal mandelic acid content was 1.4%.

It was noted that when Example 28 was smeared onto a surface, it was somewhat harder to clean than most other samples, possibly due to an interaction between the laponite and/or xanthan gum with the waxy materials.

Run 29. Acid Paste 7 was prepared by diluting Acid Paste 6 with 22 ml added water (about 30% added water) to reduce viscosity and improve blending. An oil phase was prepared using 1.50 g Softisan, 2.50 g caprylic capric triglycerides MCT, 2.10 g stearyl alcohol, 2.65 g lipstick base, 5.00 g coconut oil, 0.79 g emulsifying wax, and 200 mg caffeine. The oil phase, heated in a double boiler at about 75° C., was blended with 0.92 g of Acid Paste 7 and then after about several minutes of stirring and gradual cooling, 2.13 g of arrowroot starch was blended in at about 55° C. A smooth, slick, pleasant-feeling solid was obtained after cooling that readily dispenses against the skin, with no hint of graininess. The effective pH was estimated at about 3.2 and the mandelic acid content was nominally at least 1.9%. One problem, though, is that some clear zones apparently from Acid Paste 7 remained at the bottom of the small container used to mix the composition, suggesting that mixing was inadequate and perhaps emulsifying wax would have helped. The pH may have been lower had all the starch blended in more completely.

Run 30. The oil phase was made from 1.94 g emulsifying wax, 2.34 g beeswax, 2.60 g coconut oil, 1.51 g stearyl alcohol, 2.75 g cetyl alcohol, 2.36 g almond oil and 0.20 g caffeine. 2.31 g of Acid Paste 7 was stirred in and, after further cooling to below 60° C., about 3 g arrowroot starch was added. The nominal mandelic acid content was 3.4%.

Run 31. An oil phase was prepared using 7.00 g caprylic capric triglycerides MCT, 3.5 g stearyl alcohol, 2.00 g cacao butter, 2.21 g coconut oil, 2.5 g emulsifying wax, 1.00 g shea butter, and 200 mg caffeine. This was combined at 74° C. and then 2.12 g of Acid Paste 7 was blended in. Following slight cooling, 3.85 g of arrowroot starch was blended in. The resulting solid had a firm, smooth texture that could dispense well against the skin. Each of 3 different pH papers (0 to 14, 0 to 6, and 3.0 to 5.5) suggested the effective pH after being rubbed with water was near 3.0. The nominal mandelic acid content was 2.1%,

Run 32. An oil phase was prepared by combining 4.00 g of caprylic capric triglycerides MCT, 5.00 g cetyl alcohol, 3.50 g shea butter, 0.50 g cacao butter, 1.05 g coconut oil, and 200 mg caffeine. It was mixed at 74° C. 2.00 g of Acid Paste 7 was blended in and then, after slight cooling, 2.70 g of arrowroot starch was added. This run showed some slight separation in the bottom of the container, suggesting emulsifying wax or more intense mixing may have been helpful. The mandelic acid content was about 5.3%.

Run 33 was prepared without an added thickener from a relatively volatile solvent system. 0.398 g of mandelic acid powder was placed in a beaker with 4.11 g of 40% ethanol in water and 0.62 g of glycerin. The mandelic acid was then stirred to dissolve it completely in the solvent system. To this was added 2.60 g of cacao butter, 2.85 g of beeswax, 1.16 g of coconut oil, 1.25 g of caprylic capric triglycerides MCT, and 1.90 g of stearyl alcohol. This beaker was placed in a bath of hot water at about 80° C. After stirring, the aqueous phase was not mixing well with the oil phase, so 0.86 g of emulsifying wax was added and blended in, resulting in a good emulsion. The combination remained above 60° C. for about 5 minutes. As the combination cooled below 60° C., 3.0 g of tapioca starch was rapidly blended in with a whisk and the mixture was poured into a deodorant mold and allowed to cool. The resulting solidified material was a somewhat waxy solid with a smooth and pleasant texture having no hint of graininess. Rubbing a slight amount onto the fingers and then adding a drop of water, and touching that water to a pH strip indicated a pH around 3.

Run 34. The oil phase comprises 4.57 g stearyl alcohol, 5.35 g almond oil, 0.15 g Vitamin E, 3.58 g coconut oil, 1.12 g emulsifying wax, 1.18 g cetyl alcohol, 0.21 g glycerin, and 200 mg caffeine. During melting and mixing, the caffeine, possibly interacting with the glycerin, became gooey and clumped together. It was removed. Then 1.72 g of Acid Paste 7 were blended in with a whisk and when the temperature was below 60° C., 5.00 g of arrowroot starch was blended in. The mixture was then spooned into a 15 ml oval deodorant stick/lip balm mold and allowed to solidify. The starch concentration seemed somewhat too high for this mixture, with some evidence that portions of the acidic paste were not fully dispersed by hand stirring with a whisk.

After cooling, the solidified material was found to have a pleasant texture believed to be suitable for underarm application. The effective pH was estimated at 3.2. The nominal mandelic acid content was 2.4%,

Run 35. The oil phase was made from 5.50 g caprylic capric triglycerides MCT, 1.10 g candelilla wax, 0.18 g Vitamin E, 2.39 g cacao, 0.18 g caffeine, 0.79 g emulsifying wax, 3.1 g stearyl alcohol, 0.50 g shea butter, 1.71 g lauryl laurate, 1.53 g coconut oil, 1.52 g beeswax, and 0.25 g palmitic acid. It was prepared at about 70° C. Then 1.57 g of Acid Paste 7 was blended in, and after cooling to about 59° C., 4.91 g of arrowroot starch was blended in. This was then poured into a deodorant mold.

The effective pH was measured by taking 0.091 g of the cooled material, combining it with 0.185 g water in a tiny weighing dish about 2 cm in diameter, and thoroughly rubbing the wax and water together, followed by dipping pH papers into the water. Three pH papers were used (Hydrion 3.0 to 5.5, Hydrion 1 to 6, and a Lab Materials 1-14 paper), and all gave a pH estimate of about 3.0. The nominal mandelic acid content was 1.5%.

Run 41. Oil phase: 7.00 g behenyl alcohol, 3.53 g cacao butter, 2.00 g coconut oil, 1.51 g Milliard's emulsifying wax, 7.00 g caprylic capric triglycerides MCT, 0.58 g zinc stearate, 0.165 g magnesium myristate, 0.2 g caffeine, 1.00 g candelilla wax. This was blended with 6.33 g of Acid Paste 7 and 3.0 g arrowroot starch. The pH I measured from a slice off the side of the stick was about 4.6. The problem here was inadequate blending of the starch phase, for the reside at the bottom of the well where it was made had a pH of 2.9. But you may want to test this since the same I cut off the stick to measure 4.6 might have been influenced by residual material from the wall of the original container, so that 4.6 measurement may have been off. The nominal mandelic acid content was 4.7%.

Run 42. This one uses a new emulsifying wax, Kostol PGP, with less of the zinc fatty acid and more of the magnesium compared to Example 41. The oil phase has 7.2 g caprylic capric triglycerides MCT, 6.15 g behenyl alcohol, 1.50 g stearyl alcohol, 3.66 g cacao butter, 1.80 g coconut oil, 1.00 g Kostol PGP, 0.228 g zinc stearate, 0.282 g magnesium myristate and 0.2 g caffeine. This was blended with 5.6 g of Acid Paste 7 and 3.00 g arrowroot starch. The pH is about 3.0. The nominal mandelic acid content was 4.4%.

Run 43. The oil phase was made from 5.00 g behenyl alcohol, 8.06 g caprylic capric triglycerides MCT, 3.6 g stearyl alcohol, 3.06 g cacao butter, 2.00 g candelilla wax, 1.05 g Millard emulsifying wax, 0.128 g zinc stearate, 0.120 g magnesium myristate, and 0.2 g caffeine. The oil phase was blended with 2.85 g of Acid Paste 7 and then with 2.20 g arrowroot starch. The effective pH is about 3.1. The nominal mandelic acid content was 2.4%.

Run 44. Oil phase was made from 5.5 g FSS Sensolv (isoamyl laurate), 2.00 g emulsifying wax, 3.14 g stearyl alcohol, 3.0 g castor wax, 1.57 g behenyl alcohol, 5.08 g cacao butter, 0.27 g panthenol, 2.00 g propane diol, and 0.2 g caffeine. This was combined with 2.13 g of Acid Paste 7, and then 3.5 g of arrowroot starch was blended in and the mix was cooled. The results showed separation, possibly due to the propandiol not mixing well with the oil phase. The pH in the bulk of the mix nevertheless showed high acidity, measuring at about 2.5.

Run 44S. 25.2 g of the material were used for a second batch. 1.39 g Kostol PGP was added, with 1.00 g Softisan, 0.084 g silica dimethyl silylate, and 1.23 g Creammaker behenyl. After melting, 1.55 g tapioca starch was blended in and also 2.75 g cyclopentasiloxane. This was poured into a deodorant mold.

Run 45. The oil phase was made from 5.84 g caprylic capric triglycerides MCT, 1.08 g emulsifying wax, 2.53 g cacao butter, 2.83 g behenyl alcohol, 2.83 g stearyl alcohol, 0.183 g magnesium myristate, 0.2 g caffeine, and 0.51 g propanediol This was blended with 2.61 g Acid Paste 7 and 2.5 g arrowroot starch.

Run 46. The oil phase was made from 3.10 g caprylic capric triglycerides MCT, 1.54 g emulsifying wax, 3.4 g beeswax, 2.34 g FSS Senosolv, 5.07 g behenyl alcohol, 0.2 g caffeine, and 0.151 g magnesium myristate. This was blended with 2.54 g of Acid Paste 7 and then 2.5 g arrowroot starch.

Run 47. The oil phase comprised 0.31 g silica dimethyl silylate, 2.64 g propane diol, 4.6 g Ecosil, 1.15 g Kostol PGP, 2.59 g cacao butter, 3.72 g stearyl alcohol, 3.09 g behenyl alcohol, 0.94 g ethyl hexyl palmitate, 1.00 g almond oil, 0.2 g caffeine, and 2.81 g coconut oil. This was blended with 2.25 g Acid Paste 7 and then 2.5 g arrowroot starch. This was cooled in a deodorant mold and was found to have excellent slip with a smooth, slick texture, though a greasiness could also be detected. This deodorant was used for several days on a human subject with generally good results.

Run 47S. To 10.6 g of Example 47, add 1.28 g Creammaker Behenyl, then 0.118 g silica dimethyl silylate and 1.85 g cyclopentasiloxane, then stir in 1 drop of lavender essential oil (Radha Beauty).

Run 48. The oil phase was made from 5.65 g Ecosil, 1.07 g emulsifying wax, 5.13 g stearyl alcohol, 2.08 g castor wax, 0.2 g caffeine, and 1.86 g ethyl hexyl palmitate. This was blended with 1.58 g Acid Paste 7 and 3.0 g arrowroot starch.

Run 48S. Take 6.23 g from Example 48, melt and combine with 0.58 g Kostol PPG, 0.447 g silica dimethyl silylate, and 2.5 g cyclopentasiloxane.

Run 49. The oil phase was made from 5.00 g Ecosil, 0.50 g Kostol PGP, 2.5 g cacao butter, 0.26 g silica dimethyl silylate, 1.00 g propanediol, 2.19 g coconut oil, 1.24 g stearyl alcohol, 3.00 g behenyl alcohol, 0.2 g caffeine, and 0.33 g silica dimethyl silylate. This was blended with 5.28 g Acid Paste 7 and 2.15 g arrowroot starch. The result was too soft and did not fully harden.

Run 49S. Rework the material of Example 49 by melting it and adding 1.2 g cyclopentasiloxane.

Run 50. The oil phase was made from 8.05 g Sensosolv, 0.38 g Kostol PGP, 3.0 g stearyl alcohol, 3.00 g behenyl alcohol, 2.5 g cacao butter, 0.2 g caffeine, and 1.09 g candelilla wax. This was blended with 1.76 g Acid Paste 7 and 3.10 g arrowroot starch.

Run 51. The oil phase was made from 5.84 g almond oil, 1.33 g emulsifying wax, 4.15 g castor wax, 2.56 g behenyl alcohol, 4.30 g stearyl alcohol, 0.2 g caffeine, and 1.00 g beeswax. This was blended with 2.76 g Acid paste 7 and 2.73 g arrowroot starch. The effective pH was about 3.0.

Run 51S. Take 8.88 g remaining from Example 51, melt, and combine with 2.33 g cyclopentasiloxane, 0.31 g silica dimethyl silylate, and 2 drops lavender oil and 2 drops elemi oil.

Run 52. The oil phase was made from 4.00 g coconut oil, 1.52 g emulsifying wax, 3.90 g ethyl hexyl palmitate, 1.18 g propanediol, 3.3 g Ecosil, 1.70 g behenyl alcohol, 2.6 g stearyl alcohol, 0.2 g caffeine, and 1.73 g castor wax. This was blended with 3.18 g of Acid Paste 7 and 2.12 g arrowroot starch. There was separation of starch from the oil in this.

Run 52S. Take 8.73 g of remaining material from Example 52, melt and combine with 2.8 g cyclopentasiloxane and 0.265 g silica dimethyl silylate.

Run 53. The oil phase was made from 8.18 g caprylic capric triglycerides MCT, 2.92 g cacao butter, 3.16 g behenyl alcohol, 4.32 g stearyl alcohol, 3.80 g coconut oil, 1.85 g emulsifying wax, 0.547 g zinc stearate, 0.19 g magnesium myristate, 0.2 g caffeine, 0.19 g silica dimethyl silylate, and 1.77 g candelilla wax. 11.73 g of the melt are removed for use in Example 54. To the remaining melt, 2.01 g of Acid Paste 7 are blended in, then 3.00 g arrowroot starch. The effective pH was about 3.3.

Run 54. First 11.73 g of the oil phase of Example 53 was blended with 5.59 g cyclopentasiloxane. Then 1.8 g Acid Paste 7 and 2.28 g arrowroot starch. The effective pH was about 3.5.

Run 55. Rework 15 ml of Example 44, adding 2.06 g sunflower wax, 1.14 g behenyl alcohol, 1.75 g castor wax, and 4.78 g cyclopentasiloxane. This was then quickly poured into a mold after mixing to reduce evaporation of cyclopentasiloxane. The effective pH was about 2.9 or 3.0.

Run 56. A new Acid Paste was made using N-acetyl cysteine (NAC) instead of mandelic acid. 50 ml of water were combined with 4.40 g of corn starch and 5.0 g NAC. This was place in a 100-ml beaker and heated by microwave, stirring between brief bursts of power, to finally create a smooth, uniform paste. This is Acid Paste 8.

The oil phase was made from 7.50 g cacao butter, 2.00 g sunflower wax, 0.89 g coconut oil, 0.2 g caffeine, 1.58 g emulsifying wax, 5.03 g caprylic capric triglycerides MCT, 5.56 g stearyl alcohol, 0.66 g palmitic acid, 0.118 g magnesium myristate, and 0.297 g silica dimethyl silylate. This was blended with 2.17 g Acid Paste 8 and then with 3.00 g arrowroot starch, and then with 2 drops of elemi oil. The effective pH was about 3.5.

Run 57. The oil phase was made from 9.57 g cacao butter, 2.17 g candelilla wax, 1.39 g coconut oil, 0.3 g caffeine, 1.89 g emulsifying wax, 7.43 g caprylic capric triglycerides MCT, 5.15 g stearyl alcohol, and 1.8 g ethylhexyl palmitate. To the oil phase was added 2.06 g Acid Paste 8, 3.45 g arrowroot starch, 1 drop elemi oil and 2 drops lavender oil. After cooling, this was too soft, so it was remelted and combined with 2.00 g castor wax, 1.15 g behenyl alcohol, and 1.35 g tapioca starch.

Dimethicone Series: Three dimethicones were used with viscosities of 4200-4800 cst (DM4200), 350 cst (DM350), and 6 cst (DM6).

Acid Paste 10 was made from 3.45 g tapioca starch blended in 8.14 g water, then combined with a solution of 5.96 g mandelic acid in 36 ml of water. This was heated gradually while stirring periodically to form a viscous paste after the starch gelled.

Run 59. An oil phase was prepared using 0.385 g magnesium stearate, 0.262 g palmitic acid, 2.549 g cacao butter, 1.373 emulsifying wax, 2.39 g cetyl alcohol, 1.11 g Kostol PGP, 1.984 g stearyl alcohol, 2.747 g coconut oil, 3.59 g caprylic capric triglycerides MCT, 0.2 g caffeine, 1.43 g candelilla wax, 0.254 g silica dimethyl silylate, 3.48 g DM4200, and 1.097 g PEG-8 beeswax. This was blended with 1.8 g Acid Paste 10 and 3.15 g of arrowroot starch. The result was not uniform. It may have been the caffeine or other particles that formed a gummy substance as it interacted with the dimethicone. It did not blend as uniformly as desired.

Run 60. An oil+silicone phase was made from 5.63 g caprylic capric triglycerides MCT, 1.84 g cacao butter, 2.12 g behenyl alcohol, 1.90 g stearyl alcohol, 1.59 g emulsifying wax, 1.97 g coconut oil, 0.321 g DM4200, and 0.396 g Neoessence squalane. This was blended with 1.25 g Acid Paste 10 and 3.16 g arrowroot starch. The solidified material showed some evidence of syneresis with oily spots. The effective pH was about 3.5.

Run 61. An oil+silicone phase was made from 2.646 g behenyl alcohol, 1.50 g stearyl alcohol, 0.575 g shea butter, 2.23 g sunflower wax, 0.663 g cacao butter, 1.00 g beeswax, 2.339 g lauryl laurate, 0.611 g TKB Gelmaker, and 4.374 g caprylic capric triglycerides MCT, 1.017 g DM350, 1.562 g candelilla wax, and 0.20 g Neoessence squalane. This was blended with 3 g of Acid Paste 10 and 3.00 g arrowroot starch. The mixture was unstable and residual starch gel remained settled to the bottom after mixing.

Run 62. The oil phase was made from 5.10 g cacao butter, 2.50 g stearyl alcohol, 1.50 g behenyl alcohol, 3.27 g caprylic capric triglycerides MCT, 0.36 g ECOMulse, 0.55 g emulsifying wax, 0.45 g coconut oil, and 1.00 g candelilla wax. This was blended with 5.41 g Acid Paste 10. Then 0.520 g DM350 was blended in, followed by 4.05 g arrowroot starch. This order of addition resulted in a better, more uniform mix than in previous dimethicone trials. After pouring into a deodorant mold, the resulting product seemed somewhat soft and showed some syneresis.

Run 63. The oil phase was made from 9.28 g castor wax, 1.054 g emulsifying wax, 0.403 g ECOMulse, 1.68 g almond oil, 3.364 g caprylic capric triglycerides MCT, 2.343 g stearyl alcohol, and 1.95 g behenyl alcohol. A silicone-related phase was made by combining 0.913 g DM350, 0.574 g glycerin, 0.235 g Kostol PGP, 0.117 g ECOMulse, and 3.375 g coconut oil, then, after melting and mixing, adding 2.85 g Acid Paste 10. The oil phase was then stirred into the silicone-related phase, followed by addition of 3.10 g arrowroot starch and 4 drops elemi oil. The effective pH was about 3.2. The solid was too hard, due to excess castor wax.

Run 64. The silicone-related phase was made from 0.593 g DM350, 0.357 g DM6, 0.642 g glycerin, 0.495 g emulsifying wax, 0.662 g Neoessence squalane. After melting and mixing, 3.89 g of Acid Paste 10 was blended in. The oil phase was made from 5.20 g coconut oil, 7.95 g cacao butter, 1.10 g candelilla wax, 0.534 g Kostol PGP, 5.14 g stearyl alcohol 2.222 g behenyl alcohol, and 0.330 g silica dimethyl silylate. This was stirred into the silicone-related phase and then 4.45 g arrowroot starch was stirred in. A deodorant mold was filled. The effective pH was about 2.5.

Run 65. The oil phase was made from 0.61 g Kostol PGP, 1.50 g sunflower wax, 1.00 g candelilla wax, 1.32 g cacao butter, 1.895 g ethylhexyl palmitate, 2.73 g lauryl laurate, 2.55 g stearyl alcohol, 1.45 g behenyl alcohol, and 0.60 g Neoessence squalane.

The silicone-related phase was made from 0.25 g DM6, 0.26 g DM350, 0.27 g diphenyl siloxy phenyltrimethicone, 0.37 g glycerin, 0.10 g Kostol PGP, 0.37 g dimethicone and dimethicone/vinyl dimethicone copolymer, and 1.47 g of Acid Paste 10. After the two phases were blended at about 75° C., 2 drops of lemongrass essential oil were stirred followed by 2.07 g arrowroot starch. The effective pH was about 2.7.

Run 66. The oil phase was made from 3.50 g cacao butter, 2.00 g sunflower wax, 2.10 g stearyl alcohol, 4.04 g behenyl alcohol, 4.47 g lauryl laurate, 0.86 g ethylhexyl palmitate, 1.08 g Ecosil, 0.28 g ECOMulse.

The silicone-related phase was made from 0.123 g Kostol PGP, 0.124 g ECOMulse, 0.420 g DM350, 0.228 g DM6, 0.617 g glycerin, 0.610 g squalane, 0.567 g diphenyl siloxy phenyltrimethicone, and 1.28 g Acid Paste 10. After the two phases were blended, 3.3 g arrowroot starch was stirred in and 4 drops of spike lavender essential oil were added.

The mixture showed signs of failure as the starch separated from the oil. This was remelted and 0.40 g silica dimethyl silylate was stirred in, which helped stabilize the mixture, it seemed. The mixture was cooled to 63° C. and poured into a deodorant mold. The effective pH was about 3.0.

Run 67. The oil phase was made from 4.374 g lauryl laurate, 3.87 g sunflower wax, 0.182 g panthenol, 2.40 g ethylhexyl palmitate, 1.293 g Ecosil, 0.401 g Kostol PGP, 2.606 g behenyl alcohol, and 1.668 g stearyl alcohol.

The silicone-related phase was made from 0.630 g glycerin, 0.641 g DM6, 0.426 g DM350, 0.621 g ECOMulse, 0.096 g Kostol PGP, 0.18 g caffeine, 2.48 g Acid Paste 10. The caffeine blended well in this mixture and at least partially dissolved. After the two phases were blended, a blend of 0.342 g silica dimethyl silylate and 3.49 g arrowroot starch was stirred in. The final product appeared to have too much starch. The effective pH was about 2.5.

Run 68. The oil phase was made from 5.17 g cacao butter, 1.27 g coconut oil, 1.003 g almond oil, 3.71 g ethylhexyl palmitate, 2.145 g squalane, 0.223 g panthenol, 0.546 g Kostol PGP, 0.55 g lauryl laurate. 3.33 g stearyl alcohol, 2.96 g cetyl alcohol, 3.04 g behenyl alcohol, 0.076 g ECOMulse, and 0.183 g magnesium stearate.

The silicone-related phase was made from 1.509 g dimethicone and dimethicone/vinyl dimethicone copolymer, 0.8995 g DM350, 0.610 g DM6, 0.341 g diphenyl siloxy phenyltrimethicone, 0.805 g glycerin, 0.207 g Kostol PGP, and 3.00 g Acid Paste 10. After the two phases were blended, 3.07 g arrowroot starch was stirred in. The effective pH was about 3.4. Unfortunately, the results was too soft and showed syneresis, probably from too high silicone content.

Run 69. The oil phase was made from 4.956 g cacao butter, 1.330 g candelilla wax, 2.06 g ethylhexyl palmitate, 0.505 g squalane, 0.186 g panthenol, 0.374 g Kostol PGP, 2.47 g lauryl laurate. 2.806 g stearyl alcohol, 3.42 g behenyl alcohol, and 0.174 g magnesium stearate.

The silicone-related phase was made from 0.302 g DM350, 0.481 g glycerin, 0.042 g ECOMulse, and 0.404 g dimethicone, dimethicone/vinyl dimethicone copolymer, and about 2 g Acid Paste 10. After the two phases were blended, 2.61 g arrowroot starch was stirred in. The effective pH was about 3.4.

Run 70. The oil phase was made from 5.3 g cacao butter, 1.625 g candelilla wax, 2.55 g ethylhexyl palmitate, 2.04 g squalane, 0.233 g panthenol, 0.369 g Kostol PGP, 2.27 g lauryl laurate, 3.40 g behenyl alcohol, 0.2765 g silica dimethyl silylate, 2.96 g stearyl alcohol, and 1.47 g Ecosil.

The silicone-related phase was made from 0.772 g glycerin, 0.457 g dimethicone and dimethicone/vinyl dimethicone copolymer, 0.501 g DM350, 0.101 g Kostol PGP, and 0.067 g ECOMulse. After the two phases were blended, 2.54 g Acid Paste 10 was stirred in and after about two minutes of mixing, 3.00 g arrowroot starch was stirred in followed by 1 drop of yuzu essential oil and 2 drops of lavender essential oil. The effective pH was about 3.2.

Run 71. The oil phase was made from 2.65 g candelilla wax, 0.929 g sunflower wax, 2.07 g cacao butter, 5.11 g stearyl alcohol, 0.380 g Kostol PGP, 1.627 g Ecosil, 2.59 g lauryl laurate, 1.176 g ethylhexyl palmitate, 0.362 g squalane, and 0.943 magnesium myristate.

The silicone-related phase was made from 0.167 g DM6, 0.565 g DM350, 0.135 g Ecomulse, and 3.29 g Acid Paste 10. After blending, 1.19 g of the mix was removed and discarded to adjust the overall formulation. Then both phases were blnded and 3.30 g arrowroot starch was stirred in with 4 drops of elemi essential oil. The result was not satisfying since the solid felt too hard and had high drag against the skin.

Run 72. The oil phase was made from 5.00 g cacao butter, 0.664 g candelilla wax, 0.741 g Ecosil, 1.07 g ethylhexyl palmitate, 2.09 g lauryl laurate, 0.206 g Kostol PGP, 2.32 g stearyl alcohol, 2.000 g behenyl alcohol, and 0.910 g coconut oil.

The silicone-related phase was made from 0.876 g DM350, 0.112 g glycerin, 0.194 g Kostol PGP, 0.191 g diphenyl siloxy phenyltrimethicone, and 1.301 g Acid Paste 10. After the two phases were blended, 2.2 g arrowroot starch was stirred in with 2 drops of yuzu essential oil and 3 drops of elemi essential oil. The effective pH was about 3.0.

Run 73. The oil phase was made from 5.16 g cacao butter, 0.769 g candelilla wax, 0.752 g Ecosil, 2.314 g ethylhexyl palmitate, 0.295 g Kostol PGP, 2.377 g stearyl alcohol, and 2.055 g behenyl alcohol.

The silicone-related phase was made from 1.181 g dimethicone and dimethicone/vinyl dimethicone copolymer, 0.166 g Kostol PGP, 0.2 g caffeine, 0.222 g diphenyl siloxy phenyltrimethicone, 0.151 g glycerin, and 1.510 g Acid Paste 10. After the two phases were blended, 2.50 g arrowroot starch was stirred in. The effective pH was about 3.0.

A wide variety of other examples were also carried out. Initially demonstration of the mandelic acid stick concept was made by blending combinations of corn starch and mandelic acid to form a viscous paste with mandelic acid concentrations from 6% to 15%, using starch concentrations from about 4% to 7%. This was then blended with various oil phases, including combinations with emulsifying wax, beeswax, and other waxes, with added TKB gelmaker CC in some cases. Then, rather than stirring in starch or other solids, the mix of the oil phase and acid paste was blended in with roughly an equal part of molten commercial deodorant or antiperspirant. Some of the best results in this early phase were obtained with aluminum-free deodorants with a waxy base in which caprylic capric triglycerides were the lead ingredient, such as Women Mitchum®'s Natural Power Bamboo Powder deodorant, comprising caprylic capric triglycerides, corn starch, coconut oil, stearyl alcohol, tapioca starch, arrowroot starch, sodium bicarbonate, and other ingredients. When the acid paste was combined with the alkaline Mitchum® deodorant, the bicarbonate reacted with the mandelic acid releasing small bubbles of carbon dioxide that caused the molten mixture to foam up, changing its texture and appearance significantly, but after cooling, good results were obtained with a firm texture and, due to excess acid, a pH in the range of 3 to 4. More challenging were attempts using Native® natural deodorant and other products with larger amounts of sodium bicarbonate or in which alkaline magnesium hydroxide was the lead ingredient. Acidic mixes could be made with the starch-acid method, but the final results in some cases were not satisfying in their texture. Better results were obtained by making the composition from scratch, rather than seeking to turn non-acidic compositions into an acidic mixture.

Among the efforts to combine an acidified waxy phase with an existing deodorant stick, a silicone-based antiperspirant was used, Degree® brand from Unilever, with cyclopentasiloxane as the primary ingredient. This was melted down and combined with 1.48 g of Acid Paste 4, giving an acidic mix. However, the volatile nature of the cyclopentasiloxane resulted in mass loss from the material. But the final result, after resolidification, did not show obvious signs of failure. However, given concerns about the volatile nature of cyclopentasiloxane and other volatile silicones, in some embodiments, the inventive composition may be free or substantially free of cyclopentasiloxane and/or other volatile silicones.

### Remarks

When introducing elements of aspects of the invention or the embodiments thereof, the articles “a,” “an,” “the,” and “said” are intended to mean that there are one or more of the elements, and thus may include plural referents unless the context clearly dictates otherwise. The terms “comprising,” “including,” and “having” are intended to be inclusive and mean that there may be additional elements other than the listed elements.

Unless otherwise specified, any individual ingredient or grouping of two or more individual ingredients in a list of ingredients for any particular function may be considered optional and, in fact, may be excluded from the overall composition, as desired, and if excluded, the product or composition may be “substantially free” of that ingredient. For example, in listing aluminum silicate, silica, alumina, talc powder, and boron nitride as possible ingredient for a slip modifier, it should be understood that some embodiments may be substantially free of silica, of alumina, of talc powder, etc., or of any combination of two or more of those ingredients listed. Thus, unless otherwise indicated, a list of potential ingredients should be understood as also providing support for embodiments that exclude those ingredients or are “substantially free” of them.

Unless otherwise specified, all patents and patent applications mentioned herein should be understood to be hereby incorporated by reference to the extent they are non-contradictory herewith.

Having described aspects of the invention in detail, it will be apparent that modifications and variations are possible without departing from the scope of aspects of the invention as defined in the appended claims. As various changes could be made in the above compositions, products, and methods without departing from the scope of aspects of the invention, it is intended that all matter contained in the above description shall be interpreted as illustrative and not in a limiting sense.

While the foregoing description makes reference to particular illustrative embodiments, these examples should not be construed as limitations. The inventive system, methods, and products can be adapted for other uses or provided in other forms not explicitly listed above, and can be modified in numerous ways within the spirit of the present disclosure. Thus, the present invention is not limited to the disclosed embodiments, but is to be accorded the widest scope consistent with the claims below.

1. An acidic antiperspirant stick comprising from 1% to 25% of antiperspirants selected from zirconium and aluminum salts, from 0.5% to 10% of alpha-hydroxy carboxylic acid, 20% to 70% of lipids and/or silicone compounds, 0.5% to 10% of emulsifiers and/or gelling agents, and from 1% to 25% of a solvent that is liquid at 25° C. selected from water, alcohols, and polyols, wherein the acidic material is soluble in the solvent and stick has an effective pH from 2 to 5.

2. The acidic antiperspirant stick of claim 1, wherein the stick is substantially water free.

3. The acidic antiperspirant stick of claim 1, wherein the solvent comprises at least one of a diol and a glycol.

4. The acidic antiperspirant stick of claim 1, comprising a thickener selected from a diol, a glycol, a swellable polymer in water, a gum, a swellable mineral, and a gelatinized starch suspension in water, the thickener comprising from 0.5% to 20% of the acidic antiperspirant stick.

5. The acidic antiperspirant stick of claim 1, wherein at least 30% of the acidic matter is mandelic acid.

6. (canceled)

7. The acidic antiperspirant stick of claim 1, further comprising a sultaine-based surfactant.

8. The acidic antiperspirant stick of claim 1, having a soil penetrometer hardness of from 0.4 to 1.3 and an effective pH from 2.5 to 5.

9. The acidic antiperspirant stick of claim 1, wherein the antiperspirant comprises at least one of aluminum chlorohydrate, aluminum chloride, aluminum zirconium trichlorohydrate, aluminum zirconium tetrachlorohydrate, aluminum chlorohydrex polyethylene glycol, aluminum chlorohydrex propylene glycol, aluminum dichlorohydrate, aluminum dichlorohydrex polyethylene glycol, aluminum sesquichlorohydrate, aluminum sesquichlorohydrex polyethylene glycol aluminum sesquichlorohydrex propylene glycol aluminum zirconium octachlorohydrate, aluminum zirconium pentachlorohydrate, and aluminum zirconium tetrachlorohydrate, and has an effective pH from 2.8 to 5.

10. (canceled)

11. An acidic stick deodorant comprising a personal care composition comprising a solid or semi-solid waxy phase comprising one or more waxes, a starch or starch derivative, a thickener, and at least 1% of an alpha-hydroxy acid associated with the thickener, wherein the alpha-hydroxy acid is substantially uniformly dispersed in the stick.

12. The acidic stick of claim 11 having from 3% to 20% water, 1% to 9% alpha hydroxy acid, 5% to 35% silicone compounds, 10% to 40% lipids, 0.5% to 10% emulsifiers or gelling agents, and 2% to 20% starch or starch derivatives.

13. The acidic stick of claim 12 further comprising from 0.5% to 10% of at polyol.

14. (canceled)

15. The acidic stick of claim 11, wherein the thickener is an aqueous mixture comprising at least 0.1% of a starch or starch derivative, a swellable mineral, a gum, a water absorbing polymer, or at least 1% of a polyol having a viscosity at 25° C. of at least 5 cps.

16. The acidic stick of claim 11, wherein the stick comprises at least 3% of a thickener, the thickener comprising at least 1% of an alpha-hydroxy acid at least 5% of at least 0.5% of a starch, a gum, a polyol, or a swellable polymer.

17. (canceled)

18. The acidic stick of claim 11, having a viscosity at 25° C. of at least 100,000 cps, an effective pH between 2 and 5.5.

19. The acidic stick of claim 11, wherein the alpha-hydroxy acid is mandelic acid having a concentration from 1.5% to 9%.

20. A method for preparing an acidic stick deodorant comprising: 1) preparing a thickened aqueous phase comprising at least 3% mandelic acid and a thickener in an aqueous solution; 2) melting one or more waxes and at least one of an oil, a fatty acid, a fatty ester, a fatty alcohol, a soap, and a butter to form a molten waxy phase, 3) combining the thickened aqueous phase with the waxy phase, wherein upon cooling the mandelic acid remains substantially uniformly dispersed in the stick such that crystals of the mandelic acid cannot be readily detected when the acidic stick at 25° C. is applied to human skin.

21. The method of claim 20, further comprising 4) one or more silicone compounds that make up from 3% to 40% of the acidic stick, and 5) combining a powder with one of the waxy phase, the combination of the aqueous phase with the waxy phase, or the combination of the waxy phase and the silicone compounds, or the combination of the waxy phase with the silicone compounds and the aqueous phase.

22. (canceled)

23. The method of claim 20, wherein the acidic stick comprises from 3% to 15% water, 0.5% to 7% mandelic acid, at least 2% of a silicone liquid, from 10% to 40% lipids, at least 5% starch, and at least 1% of a polyol liquid.

24. (canceled)

25. The method of claim 20, wherein the thickener comprises water, a polyol, and at least 3% of mandelic acid.

26. (canceled)

27. The method of claim 20, further comprising combining caffeine with at least one of the aqueous phase and the oil phase.

28. (canceled)

29. (canceled)
